(19) United States
(12) Patent Application Publication
(10) Pub. No.: US 2026/0016484 A1
(43) Pub. Date: Jan. 15, 2026

(54) SUPEROXIDE-RESPONSIVE QUINONE METHIDE PRECURSOR (QMP-SO) AND METHOD OF USE

(71) Applicants: The University of Hong Kong, Hong Kong (HK); Centre for Oncology and Immunology Limited, Hong Kong (HK)

(72) Inventors: Yik Sham Clive CHUNG, Hong Kong (HK); Hin Yuk LAI, Hong Kong (HK)

(21) Appl. No.: 19/264,910

(22) Filed: Jul. 10, 2025

Related U.S. Application Data
(60) Provisional application No. 63/669,255, filed on Jul. 10, 2024.

(51) Int. Cl. G01N 33/68; G01N 33/50; G01N 33/58

(52) U.S. Cl. CPC G01N 33/6848; G01N 33/5005; G01N 33/582

(57) ABSTRACT

The present invention discloses superoxide-specific quinone methide precursors (QMP-SOs) that enable proximity labeling and chemoproteomics to investigate superoxide redox biology. QMP-SOs are activated by superoxide to generate a reactive quinone methide intermediate that covalently tags nearby proteins. These probes exhibit high selectivity toward superoxide over other ROS, enabling protein labeling in superoxide-rich cellular compartments. QMP-SOs are compatible with fluorescence imaging and tandem mass tag (TMT)-based mass spectrometry, facilitating visualization of superoxide dynamics and identification of superoxide-regulated proteins. In menadione-treated HepG2 cells, QMP-SO-TMT profiling identified mitochondrial proteins including DJ-1 and DLDH as redox-sensitive targets, linking superoxide stress to cell survival and metabolism. The QMP-SO platform uniquely combines spatial proximity labeling with superoxide specificity, allowing previously inaccessible insights into dynamic and localized redox signaling. This invention is broadly applicable in studying superoxide-mediated processes and holds potential for therapeutic target discovery in diseases driven by oxidative stress, including cancer, aging, and neurodegeneration.

### CROSS-REFERENCE TO RELATED APPLICATIONS

The present application claims the priorities from the U.S. provisional patent application Ser. No. 63/669,255 filed Jul. 10, 2024, and the disclosure of which is incorporated herein by reference in its entirety.

### FIELD OF THE INVENTION

The present invention generally relates to at least the fields of chemical biology, molecular biology, and bioanalytical chemistry; specifically focusing on the development of superoxide-responsive quinone methide precursors (QMP-SOs) for studying superoxide biology through proximity labeling and chemoproteomics.

### BACKGROUND OF THE INVENTION

Superoxide (O₂·⁻) is a major source of reactive oxygen species (ROS) in biological systems and is known to be involved in key pathological processes, including innate immune responses. Emerging evidence indicates that superoxide, along with other ROS such as hydrogen peroxide and hydroxyl radicals, plays essential roles in physiological functions and serves as a signaling molecule when its production is tightly regulated. By inducing redox-dependent post-translational modifications, superoxide can modulate the activity and function of biomolecules, thereby participating in a broad range of cellular signaling pathways. However, excessive or uncontrolled generation of superoxide results in oxidative stress, which has been implicated in the onset and progression of various diseases, including cancer, aging-related disorders, and neurodegenerative conditions. Consequently, the ability to monitor superoxide levels and to identify biomolecules involved in redox signaling is vital for advancing the understanding of superoxide biology and its relevance to human health.

Various superoxide-specific fluorescent probes have been developed to facilitate the detection of superoxide in biological samples. These probes typically exhibit a “turn-on” fluorescence response upon reacting with superoxide, thereby enabling visualization and monitoring of superoxide distribution in cells or tissues with high spatial and temporal resolution through fluorescence imaging. However, as illustrated in FIG. 1A, such probes are limited to visual detection and cannot directly identify the biomolecules that are redox-modified in response to superoxide. To investigate oxidative post-translational modifications on proteins, chemoproteomic probes have been employed as analytical tools. Despite their effectiveness in labeling oxidized proteins, these probes generally lack specificity for the reactive oxygen species responsible for the modification events. Consequently, they do not provide insight into which ROS mediates the observed modifications or how such redox processes are initiated and regulated. The signaling pathways governed by superoxide remain poorly understood, largely because superoxide-induced modifications are reversible, short-lived, and highly dynamic. These characteristics make them difficult to capture using traditional biochemical or biological methods.

Superoxide is characterized by its high reactivity, short half-life, and limited diffusion capacity in biological environments. In contrast, other reactive oxygen species such as hydrogen peroxide exhibit greater stability and broader diffusion. Given these properties, chemical probes designed to detect or label superoxide should ideally react in close proximity to the sites of superoxide generation—commonly referred to as superoxide hotspots—to avoid non-specific or off-target labeling. For this purpose, quinone methide represents a highly suitable functional group, as it possesses strong electrophilicity and readily reacts with nucleophilic amino acid residues on nearby proteins, making it an effective moiety for proximity-based covalent labeling.

Currently there are no superoxide-specific molecular probes available for profiling proteins associated with superoxide biology. This greatly hinders the understanding of superoxide redox biology, which are highly dynamic and difficult to be studied by conventional biochemical/biological experiments.

Several references have disclosed ROS-responsive probes for imaging or labeling purposes; however, these systems primarily target hydrogen peroxide or lack the capability for superoxide-specific, proximity-based covalent labeling required for proteomic analysis. For instance, US 20230384315 A1 describes a probe system responsive to hydrogen peroxide for activity-based sensing and labeling, while US 20210061842 A1 discloses a puromycin-based probe for general ROS detection, without specificity toward superoxide. US 20240018174 A1 presents quinone methide analogs for labeling phosphatases or in vitro enzyme targets, but not for use in a superoxide-responsive context. Similarly, probes for senescence detection (WO 2023183328 A2) and H₂O₂-responsive labeling (e.g., US 20210096142 A1, WO 2017033163 A1) are not designed to react selectively with superoxide. Although US 20210096142 A1 mentions a probe capable of detecting superoxide anion radicals, it is limited to fluorescence imaging and does not support covalent labeling or protein profiling. To date, no known probe combines superoxide-specific reactivity with electrophilic tagging functionality for proximity labeling and chemoproteomic applications. This demonstrates a clear gap in the current state of the art, highlighting the need for the present invention.

### SUMMARY OF THE INVENTION

Currently, there is no commercially available probe capable of studying superoxide by chemoproteomics and mass spectrometry. The present invention aims to develop superoxide-specific probes that can work with MS and chemoproteomics experiments to identify proteins that are targets of superoxide redox biology.

The present invention relates to the design and synthesis of superoxide-responsive quinone methide precursors, QMP-SOs (FIG. 1B). Liquid chromatography-mass spectrometry (LC-MS) experiments revealed the good reactivity and selectivity of QMP-SO toward superoxide over other ROS in aqueous buffer solutions. Shotgun mass spectrometry confirmed that QMP-SOs are capable of introducing a covalent tag onto proteins in a superoxide-dependent manner, enabling proximity labeling of proteins within superoxide-rich microenvironments.

Further applications of QMP-SOs in fluorescence imaging enabled the detection of superoxide in cells under oxidative stress. Importantly, QMP-SO could couple with tandem mass tag (TMT) to perform mass spectrometry (MS)-based chemoproteomics experiment, herein referred to as QMP-SO-TMT. This method allowed the proteome-wide profiling of superoxide-regulated proteins in cells treated with superoxide inducers such as menadione, which stimulates mitochondrial superoxide production. DJ-1 and dihydrolipoamide dehydrogenase (DLDH) are two of the proteins identified by QMP-SO-TMT, providing insights into the interplay of cell survival, autophagy, cell death and cell metabolism with superoxide redox biology. It exhibits time-dependent oxidative modification at Cys106. This modification was associated with the activation or inactivation of survival pathways (MEK/Erk, Akt) depending on the oxidative stress duration. In addition, DLDH, a mitochondrial enzyme essential for cellular metabolism, was identified as a superoxide-modified protein with impaired enzymatic activity. These findings highlight the capability of QMP-SOs to uncover functional oxidative modifications on key proteins, elucidating how superoxide signaling governs cellular stress responses, metabolic adaptation, and apoptosis.

There are fluorescent probes available for detecting superoxide levels in biology samples by fluorescence imaging. However, they cannot be applied for chemoproteomics or mass spectrometry (MS) experiments to identify proteins associated with superoxide biology. In contrast, the QMP-SO probes described herein uniquely enable chemoproteomics-based profiling of superoxide-associated protein targets. These proteins may serve as biomarkers or druggable targets in diseases driven by oxidative stress, including cancer, aging, and neurodegenerative disorders. The QMP-SO platform is therefore expected to have broad value in both academic research and pharmaceutical development.

In a first aspect, the present invention provides a composition having a superoxide-reactive probe of formula (I):
QMP-SO-R¹, or a structural analog, derivative, or pharmaceutically acceptable salt thereof,

QMP-SO includes a quinone methide precursor moiety having a phenol ring substituted at a para-position with a superoxide-reactive trigger group, and a hydroxyl group of the phenol ring is covalently bonded to a linker. The linker includes R¹ selected from an alkyne group, an azide group, a biotin, or a fluorophore. The composition is configured to generate a para-quinone methide intermediate upon reaction with superoxide under aqueous physiological conditions, and the para-quinone methide intermediate is capable of covalently labeling nucleophilic residues on nearby proteins.

In accordance with one embodiment, the R¹ is an alkyne group suitable for Cu(I)-catalyzed azide-alkyne cycloaddition (CuAAC) reaction.

In accordance with one embodiment, the superoxide-reactive trigger group includes diphenylphosphonate or triflate.

In accordance with one embodiment, the quinone methide intermediate selectively reacts with nucleophilic amino acid residues selected from the group consisting of cysteine, lysine, histidine, tyrosine, serine, threonine, glutamic acid, and aspartic acid.

In accordance with one embodiment, the superoxide-reactive probe exhibits minimal reactivity toward other reactive oxygen or nitrogen species, including hydrogen peroxide, hypochlorous acid, nitric oxide, and peroxynitrite.

In accordance with one embodiment, the composition further includes a pharmaceutically acceptable carrier suitable for delivery to cells or tissues.

In accordance with one embodiment, the R¹ is conjugated to a reporter moiety selected from a fluorophore or desthiobiotin via CuAAC.

In a second aspect, the present invention provides a method for labeling superoxide-associated proteins in a biological sample, including contacting the biological sample with a composition of claim 1, wherein a superoxide-reactive probe in the composition is present in a concentration in a range of about 0.1 μM to 100 μM; reacting the superoxide-reactive probe with endogenously or exogenously generated superoxide to form a quinone methide intermediate; and covalently labeling proteins proximal to superoxide hotspots via electrophilic addition of the quinone methide intermediate to nucleophilic amino acid residues.

In accordance with one embodiment, the method further includes subjecting the labeled proteins to Cu(I)-catalyzed azide-alkyne cycloaddition (CuAAC) reaction with a detection reagent.

In accordance with one embodiment, the detection reagent includes a fluorophore or a biotin moiety.

In accordance with one embodiment, the biological sample includes a population of cells, tissue, an organoid, or a cell lysate derived from a mammal.

In accordance with one embodiment, the method further includes treating the biological sample with a superoxide-inducing agent selected from menadione, antimycin A, or xanthine/xanthine oxidase.

In accordance with one embodiment, the labeled proteins include DJ-1, and a residue of Cys106 of the DJ-1 is oxidatively modified in a superoxide-dependent manner.

In accordance with one embodiment, the labeled proteins include dihydrolipoamide dehydrogenase (DLDH), and the labeling occurs at one or more nucleophilic residues within DLDH, resulting from a superoxide-triggered para-quinone methide reaction.

In accordance with one embodiment, the biological sample is further treated with an antioxidant selected from N-acetylcysteine (NAC) or MnTBAP.

In a third aspect, the present invention provides a kit for detecting superoxide-associated protein labeling. The kit includes a detection reagent comprising a fluorophore or biotin moiety, instructions for performing superoxide-dependent protein labeling, and a composition having a superoxide-reactive probe of formula (I):
QMP-SO-R¹, or a structural analog, derivative, or pharmaceutically acceptable salt thereof.

In accordance with one embodiment, the kit further includes a superoxide-inducing agent selected from menadione or antimycin A and a superoxide scavenger selected from MnTBAP or N-acetylcysteine.

In accordance with one embodiment, the detection reagent includes a fluorophore or desthiobiotin moiety conjugated to an azide-functionalized polyethylene glycol (PEG) linker, formulated in an aqueous buffer comprising a copper(I)-stabilizing ligand selected from tris(benzyltriazolylmethyl)amine (TBTA) or bathophenanthroline disulfonic acid (BPDS), thereby enabling efficient Cu(I)-catalyzed azide-alkyne cycloaddition (CuAAC) under physiological pH and temperature conditions.

In accordance with one embodiment, the kit further includes a lyophilized control reagent comprising a purified protein covalently labeled at a defined nucleophilic residue by the superoxide-reactive probe, and packaged with reference SDS-PAGE and/or mass spectrometry data.

In accordance with one embodiment, the superoxide-reactive probe is provided at a concentration of 0.1 μM to 100 μM; the detection reagent is provided at a concentration of at least 1 μM, and the kit components are arranged in a multi-well plate or vial-based format.

### BRIEF DESCRIPTION OF THE DRAWINGS

Embodiments of the invention are described in more details hereinafter with reference to the drawings, in which:

FIG. 1A shows examples of reported superoxide probes and their applications in studying superoxide; FIG. 1B shows chemical structures of QMP-SOs reported in this invention. The alkyne handle enables conjugation to fluorophore or desthiobiotin through CuAAC reaction for detection and analysis; FIG. 1C shows schematic cartoon illustrating the working principle of QMP-SOs for proximal protein labeling within or adjacent to superoxide-rich compartments, i.e., superoxide hotspots; this allows monitoring superoxide level in biological samples by fluorescence imaging and identification of proteins associated with superoxide biology by LC-MS/MS;

FIG. 2A shows schematic cartoon illustrating the LC-MS experiments to study reactivity of QMP-SO toward redox-active species in aqueous buffer solution; FIGS. 2B-2C depict Selected-ion chromatograms (SIC), corresponding to the molecular ions from QMP-SO-C3-alkyne and QMP-SO-C5-alkyne respectively, were measured from aliquots of the reaction mixture of QMP-SO-C3-alkyne/QMP-SO-C5-alkyne and KO₂ in PBS-MeOH mixture (4:1, v/v) at the indicated time; FIG. 2D depicts percentage of unreacted QMP-SOs in the solution mixture with KO₂; FIG. 2E depicts equations illustrating the generation of superoxide (O₂·⁻) by the xanthine and xanthine oxidase (XOD) system, and the scavenging of superoxide by superoxide dismutase (SOD); FIG. 2F depicts percentage of QMP-SO-C5-alkyne reacted in the solution mixture containing xanthine and XOD; FIG. 2G depicts percentage of QMP-SO probes reacted in the presence of xanthine, XDO and/or SOD; FIG. 2H depicts percentage of QMP-SO-C5-alkyne reacted after incubation with the redox-active species for 30 min in the aqueous buffer solution; FIG. 2I depicts stability of QMP-SOs in PBS-MeOH mixture (4:1, v/v); quantified data were shown in average±SD; statistical analysis using a two-tailed Student's t-test; ***P<0.001 and ****P<0.0001;

FIG. 3 shows percentage of unreacted QMP-SO-C5-alkyne in the solution mixture with KO₂ at different pHs, as measured by LC-MS experiments after incubation for 15 min; the slightly higher amount of unreacted probe at pH 6.34 can be attributable to the lower stability of KO₂ in acidic buffer solutions, resulting in less superoxide to react QMP-SO-C5-alkyne;

FIG. 4A shows schematic cartoon illustrating the gel-based chemoproteomics experiments to identify QMP-SO-C5-alkyne-induced modifications on a model protein, bovine serum albumin (BSA); FIG. 4B depicts in-gel fluorescence from BSA incubated with QMP-SO-C5-alkyne, KO₂ and/or SOD in an aqueous buffer solution; FIG. 4C depicts schematic cartoon illustrating the LC-MS/MS experiments to identify QMP-SO-1-induced modifications on BSA; FIG. 4D depicts the number of QMP-SO-1-induced modifications on BSA in PBS with or without addition of KO₂; FIG. 4E depicts representative MS/MS revealing the covalent modification of Glu (E*) of BSA by QMP-SO-1; FIG. 4F depicts the number of modified and unmodified amino acids on BSA by QMP-SO-1; FIG. 4G shows the sites of QMP-SO-1-induced modifications on BSA, with the same labeling color as that in FIG. 4F; quantified data were shown in average±SD (n=2 replicates/group); statistical analysis using a two-tailed Student's t-test; **P<0.01 and ***P<0.001;

FIGS. 5A-5B are representative MS/MS showing the QMP-SO-1-induced modifications on Cys and Asp of BSA respectively;

FIGS. 6A-6C are representative MS/MS showing the QMP-SO-1-induced modifications on His, Lys and Arg of BSA respectively;

FIGS. 7A-7C are representative MS/MS showing the QMP-SO-1-induced modifications on Ser, Thr and Tyr of BSA respectively;

FIG. 8 shows the number of modified and unmodified nucleophilic amino acids on BSA by QMP-SO-1 in the presence of KO₂, as found in the shotgun MS experiment;

FIG. 9A shows in-gel fluorescence analysis of HepG2 cell lysates (50 μg) incubated with QMP-SO-C5-alkyne (10 μM), KO₂ (500 μM), and/or SOD (75 mU/mL) in PBS for 2 h. Proteins were precipitated with acetone (−20° C., overnight), re-dissolved in PBS, reacted with azide-fluor 545 (25 μM) via CuAAC, and analyzed by SDS-PAGE; FIG. 9B shows in-gel fluorescence analysis of HepG2 cell lysates obtained from live cells treated with QMP-SO-C5-alkyne (10 μM), menadione, and/or N-acetylcysteine (NAC; 5 mM) for 3 h. Lysates were reacted with azide-fluor 545 and analyzed as in FIG. 9A; FIG. 9C shows confocal fluorescence imaging of live HepG2 cells treated with QMP-SO-C5-alkyne (10 μM) and the indicated reagents for 3 h; cells were fixed, permeabilized, reacted with azide-fluor 545 via CuAAC, stained with Hoechst (8.2 μM), and imaged; FIGS. 9D-9E depict quantification of the in-gel fluorescence intensity from FIGS. 9A and 9B respectively; n=3 replicates/group; FIG. 9F depicts quantification of confocal fluorescence intensities from FIG. 9C (n=30 cells from 3 biological replicates/group); quantified data were shown in average±SD; statistical analysis using a two-tailed Student's t-test; *P<0.05, ***P<0.001 and ****P<0.0001; ns=not significant;

FIG. 10 shows MTT assay for studying the viability of HepG2 cells incubated with QMP-SO-C3-alkyne and QMP-SO-C5-alkyne in complete medium for 4 h;

FIG. 11 shows in-gel fluorescence analysis of HepG2 cell lysates following treatment with QMP-SO-C5-alkyne (10 μM) and menadione, with or without MnTBAP (200 μM); cells were pretreated with MnTBAP or vehicle control for 12 h, followed by a 4 h co-incubation with QMP-SO-C5-alkyne and the indicated reagents; after PBS washing and sonication, lysates were protein-normalized, labeled with azide-fluor 545 (25 μM) via CuAAC, and subjected to SDS-PAGE. Labeled proteins were visualized by in-gel fluorescence; quantified data were shown in average±SD (n=3 replicates/group); statistical analysis using a two-tailed Student's t-test; **P<0.01 and ****P<0.0001;

FIG. 12A shows schematic cartoon illustrating the workflow of the MS experiment to identify proteins labeled by QMP-SO-C5-alkyne; FIG. 12B depicts volcano plot showing the MS result from HepG2 cells treated with menadione (100 μM) for 2 h; the dots are the proteins enriched in the menadione-treated sample (>2-fold) with statistical significance (p<0.05) and annotation to the oxidation-reduction process (Gene ontology biological process: 0055114); FIG. 12C depicts gene ontology (GO) analysis of the cellular component of the enriched proteins; FIG. 12D depicts PANTHER GO-Slim analysis of the biological process of the enriched proteins;

FIG. 13A shows immunoblotting of cells treated with menadione and SOD mimetic, MnTBAP, for 2 h at the indicated concentrations; FIGS. 13B-13C show immunoblotting of cells treated with menadione at the indicated concentrations and time intervals, in the absence or presence of NAC; FIG. 13D shows schematic cartoon illustrating the roles of DJ-1 in regulating cell survival and cell death signals in cells facing superoxide stress from menadione treatment; FIG. 13E depicts DLDH activity assay by monitoring UV-vis absorption spectra of mitochondria extract from cells treated with DMSO or menadione, dihydrolipoamide (3 mM), EDTA (1.5 mM) and NAD⁺ (3 mM) in potassium phosphate (100 mM, pH 8.0); FIG. 13F depicts changes in UV-vis absorption at 340 nm over time from the solution mixture of mitochondria extract, dihydrolipoamide, EDTA and NAD⁺; FIG. 13G depicts UV-vis absorption at 340 nm of the solution mixture after incubation for 180 s; FIG. 13H depicts Schematic cartoon illustrating the roles of DLDH and its oxidation in governing lipoamide dehydrogenation; quantified data were shown in average±SD (n=3 replicates/group); statistical analysis using a two-tailed Student's t-test; **P<0.01, ***P<0.001 and ****P<0.0001; and

FIG. 14 shows immunoblotting to investigate levels of mitochondrial, cytosolic, endoplasmic reticulum and nucleus markers, COX IV, GAPDH, calreticulin and lamin A/C respectively, in the mitochondrial extract of HepG2 cells.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “superoxide-reactive probe” refers to a molecule or compound comprising a chemical structure that selectively reacts with superoxide (O₂·⁻) under aqueous physiological conditions. The reaction results in the conversion of the probe into a reactive intermediate, such as a para-quinone methide, that enables covalent labeling of target biomolecules, including proteins. The superoxide-reactive probe may comprise a superoxide-reactive trigger group and a conjugatable moiety.

As used herein, the term “quinone methide precursor” refers to a chemical moiety that, upon specific activation such as reaction with superoxide, generates a quinone methide intermediate. The precursor typically includes a phenol ring substituted with a superoxide-reactive group at the para-position relative to the hydroxyl group.

As used herein, the term “superoxide-reactive trigger group” refers to a functional group that is cleaved or transformed in the presence of superoxide under physiological conditions.

As used herein, the term “linker” refers to a covalent moiety connecting the reactive portion of the probe (e.g., the quinone methide precursor) to a functional group, denoted as R¹. R¹ may be selected from a fluorophore, biotin, desthiobiotin, an alkyne group, an azide group, or a combination thereof. The linker may be linear, branched, or polyethylene glycol-based.

As used herein, the term “para-quinone methide intermediate” refers to a reactive electrophilic species bearing a para-oriented quinone methide structure, which is formed upon elimination or transformation of the precursor moiety in the presence of superoxide. The intermediate is capable of undergoing covalent addition to nucleophilic residues in biomolecules such as proteins.

As used herein, the term “nucleophilic residues” refers to amino acid side chains within proteins that possess nucleophilic functional groups capable of reacting with electrophilic species such as quinone methide.

As used herein, the term “detection reagent” refers to a molecule that is capable of conjugating to a reactive group such as an alkyne or azide via CuAAC reaction.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

In the following description, QMP-SOs are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Superoxide is highly reactive, short-lived, and has a small diffusion radius in biological systems, while other ROS, such as H₂O₂, have higher biostability. This suggests that the proteins redox-modified by superoxide should be in close proximity to where superoxide is generated endogenously, inspiring the development of new chemical probes to induce proximity labeling of proteins in superoxide-rich compartments, i.e. superoxide hotspots, to profile proteins associated with superoxide biology. The chemical probes should tag onto nearby proteins for analysis without diffusing away from the superoxide hotspots to prevent off-target labeling. Quinone methide, which is highly electrophilic and reacts readily with nucleophilic amino acids on proximal proteins, should be an ideal moiety for proximity labeling.

Therefore, the present invention provides a class of chemical probes, termed superoxide-responsive quinone methide precursor (QMP-SO) and its derivatives, which are designed for covalent labeling of proteins located in proximity to superoxide generation sites within biological systems.

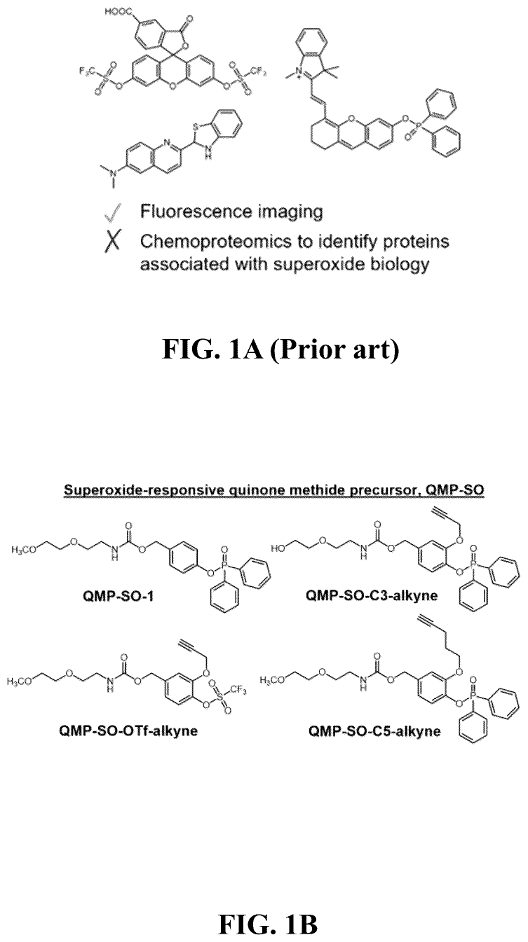

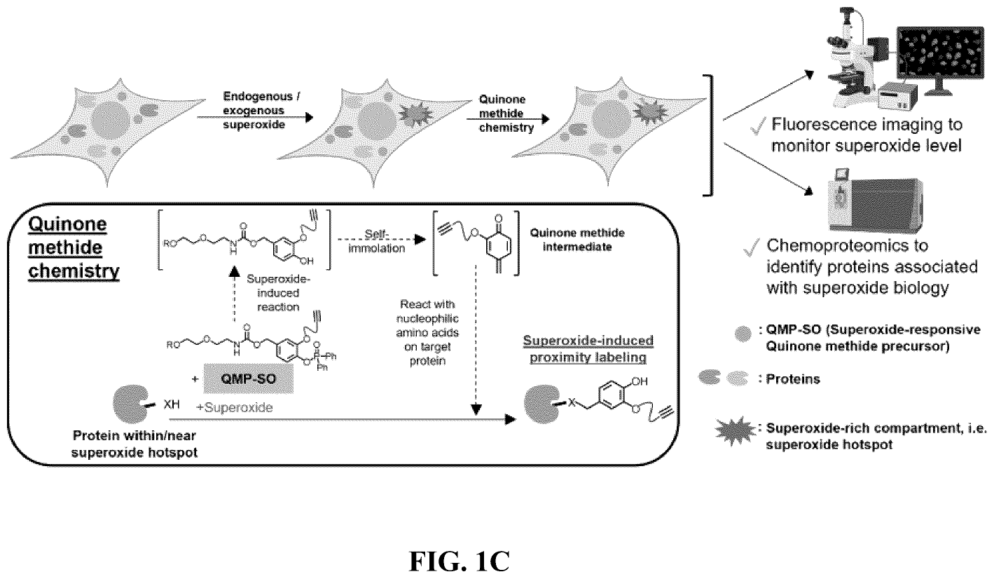

QMP-SOs are superoxide-specific probes capable of studying superoxide biology through fluorescence imaging and chemoproteomics. The probes contain a superoxide-responsive trigger which remains intact in the absence of superoxide. Upon exposure to superoxide, the trigger would be activated and cleaved off, forming the 4-hydrobenzyl carbamate intermediate. Subsequent self-immolation through the 1,6-elimination reaction generates the highly electrophilic quinone methide, which reacts readily with nucleophilic amino acids on proximal proteins and hence induces a covalent tag onto the proteins (FIG. 1C). Further functionalization of the probes with an alkyne handle allows downstream analysis of the tagged protein through the installation of a fluorophore or desthiobiotin by the CuAAC reaction.

The QMP-SOs are designed with a para-substituted phenol moiety bearing an electron-withdrawing trigger group (e.g., diphenylphosphonate or triflate) and an alkyne handle for subsequent bioconjugation. Upon reaction with superoxide, the trigger is eliminated via a 1,6-elimination mechanism to generate a para-quinone methide (QM) intermediate. This electrophilic intermediate enables covalent labeling of nucleophilic amino acid residues on proteins located in the vicinity of the superoxide source.

The QMP-SO probe is designed to achieve selective superoxide activation, electrophilic reactivity, and proximity-based labeling, enabling both fluorescence imaging and proteomic analysis of superoxide-associated biomolecules.

The QMP-SOs contain a terminal alkyne group that allows the introduction of fluorophores or affinity tags via CuAAC reaction. The present invention successfully synthesized QMP-SO-1 and three alkyne-containing probes, namely QMP-SO-OTf-alkyne, QMP-SO-C3-alkyne and QMP-SO-C5-alkyne (FIG. 1B). QMP-SO-OTf-alkyne contains a triflate group as the superoxide-responsive trigger, while the others utilize diphenylphosphonate as the trigger.

The alkyne probes were synthesized by first installing the alkyne moiety onto the meta-position of 3,4-dihydroxybenzaldehyde through a nucleophilic substitution reaction, followed by incorporating the trigger onto the para-position. Subsequent aldehyde reduction, activation of the hydroxyl group by disuccinimidyl carbonate and reaction with amine-functionalized glycol yielded the final products. All the compounds have been successfully characterized by ¹H, ¹³C, ¹⁹F and/or ³¹P NMR (ESI), and LC-MS.

Through mass spectrometry (MS) and chemoproteomics experiments, QMP-SO has successfully uncovered the roles of proteins regulated by superoxide redox biology to govern important cellular processes including cell proliferation and apoptosis, cell metabolism and autophagy.

Due to the high reactivity, short lifetime, and limited diffusion radius of superoxide in biological systems, proteins modified by the QMP-SO probes are likely located within or proximal to superoxide-enriched microenvironments, herein referred to as “superoxide hotspots.” The generated QM intermediate reacts broadly with various nucleophilic side chains such as Cys, Lys, His, Arg, and others, as demonstrated by shotgun MS analysis, enabling the primary-sequence-independent tagging of proteins.

This method is the first to induce proximity labeling of proteins in a superoxide-dependent manner, thus enabling the detection of superoxide in biological samples by fluorescence imaging and, more importantly, profiling proteins associated with superoxide redox biology by MS and chemoproteomics experiments, where the later has not been reported in the literature. The platform further supports multiplexed quantitative proteomics via TMT labeling, enabling system-level identification and comparison of redox-sensitive proteins across different conditions.

It is anticipated that the QMP-SO probes can be applied in different biological models to discover new redox biology and protein targets of superoxide. These proteins should be potential drug targets for the development of new therapy, as oxidative stress is known to be associated with the development and propagation of many diseases such as cancers, aging and neurodegenerative disorders.

In the following description, specific details are provided to offer a comprehensive understanding of the present invention, for explanatory purposes and not intended for limitation.

### EXAMPLE

### Example 1—Materials and Methods

### LC-MS Experiments to Investigate Reactivity and Selectivity of QMP-SOs

A stock solution of QMP-SOs in DMSO was diluted by PBS/MeOH solution mixture (1:1, v/v) to a final concentration of 100 μM. The stock solution of ROS was freshly prepared and added to the compound solution at a final concentration of 500 μM. The solution mixture was incubated at 37° C. for 60 min. After incubation, the reaction mixture (50 μL) was diluted by PBS/MeOH (1:1, v/v; 450 μL) and 10 μL of the diluted solution was sent for LC-MS analysis on Waters Autopurification System using a SunFire C18 HPLC column (50×4.6 mm with 5 μm diameter particles, Waters). Separation was achieved by gradient elution from 5% to 100% MeCN in water (constant 0.1 vol % formic acid) over 4 min, isocratic elution with 100% MeCN (with 0.1 vol % formic acid) from 4 to 8 min, and returning to 5% MeCN in water (with 0.1 vol % formic acid) and equilibrated for 2 min. The selected ion chromatogram, with m/z corresponding to the molecular ion of QMP-SO, was extracted.

The data was analyzed using MassLynx™ software by determining the area under the curve. The peak area was recorded to calculate the percentage change of QMP-SO after ROS incubation. The ROS stock solution was prepared as follows: O₂·⁻ was generated from KO₂. The concentration was determined by UV absorption at 256 nm (molar extinction coefficient=2686 M⁻¹cm⁻¹) using UV-vis absorption spectrometry. OCl⁻ was generated from NaOCl (˜4% w/w) purchased from Macklin. ·OH was generated through the Fenton reaction using (NH₄)₂Fe(SO₄)₂·6H₂O solution and H₂O₂. (CH₃)₃COO· was generated from the Fenton reaction between (NH₄)₂Fe(SO₄)₂·6H₂O and (CH₃)₃COOH. NO· was generated from NONOate solution diluted in 10 mM NaOH. The concentration was determined by UV absorption at 252 nm (molar extinction coefficient=8400 M⁻¹cm⁻¹) using UV-vis absorption spectrometry. Peroxynitrite was purchased from Calbiochem. The concentration was determined by UV-vis absorption at 302 nm (molar extinction coefficient=1670 M⁻¹ cm⁻¹) using UV-vis absorption spectrometry.

Reaction of QMP-SOs with O₂·⁻ Generated from Xanthine/Xanthine Oxidase (XOD) System

For O₂·⁻ generated from xanthine and xanthine oxidase, XOD was premixed with QMP-SO-C5-alkyne at final concentration of 0.08 U/mL and 5 μM respectively, before the addition of xanthine at the indicated concentration. For samples co-incubated with SOD, SOD was premixed with XOD at final concentration of 75 mU/mL before addition of xanthine. After 30 min incubation, an aliquot of the reaction mixture (10 μL) was sent for LC-MS analysis as described previously. Selected ion chromatograms were extracted and analyzed by integrating the area under curve.

### In-Gel Fluorescence Assay of Protein Labeling on BSA by QMP-SO-1

50 μL BSA (2 mg/mL) in PBS was incubated with QMP-SO-C5-alkyne (10 μM) and KO₂ (500 μM) in the presence or absence of SOD (150 mU/mL) for 1 h. After 1 h incubation at room temperature, proteins were precipitated with pre-chilled acetone (300 μL) at −20° C. for 4 h. The samples were centrifuged at 5,000 g at 4° C. for 10 min, and the supernatant was discarded. The protein pellets were washed with pre-chilled 0.01M HCl/90% acetone, then methanol, and re-suspended in 200 μL PBS. The protein samples were further diluted in PBS (sample:PBS=1:49, v/v). A master mix for CuAAC was prepared from azide-fluor 545 (1 mM), copper (II) sulfate (50 mM), THPTA (50 mM) and freshly prepared sodium ascorbate (250 mM) and added to 50 μL of the lysates with the final concentrations of azide-fluor 545, copper(II) sulfate, THPTA and sodium ascorbate in the solution mixture at 5 μM, 1 mM, 3 mM and 5 mM respectively. The solution was incubated in dark at room temperature for 1 h, quenched with 4× reducing Laemmli SDS sample loading buffer and boiled at 90° C. for 5 min. Samples were then separated by molecular weight on precast FuturePAGE 4-20% gels and imaged by ChemiDoc MP (Bio-Rad Laboratories, Inc) for measuring in-gel fluorescence. The protein loading was determined by Pierce™ Silver Stain Kit (Thermo Fisher Scientific, #24612).

### In-Gel Fluorescence Assay of Protein Labeling in HepG2 Cell Lysates by QMP-SO-C5-Alkyne

HepG2 cell lysates were extracted with PBS by probe sonication. 50 μL cell lysates (2 mg/mL) in PBS were incubated with QMP-SO-C5-alkyne (10 μM) and indicated concentrations of KO₂ (0, 250 or 500 μM) in the presence or absence of GSH (10 mM) or SOD (75 mU/mL) for 1 h. After 1 h incubation at room temperature, proteins were precipitated with pre-chilled acetone (300 μL) at −20° C. for 4 h. The samples were centrifuged at 5,000 g at 4° C. for 10 min, and the supernatant was discarded. The protein pellets were washed twice with pre-chilled 0.01M HCl/90% acetone, then methanol, and re-suspended in 50 μL PBS. The azide-fluor 545 was conjugated to QMP-SO-labelled proteins through the CuAAC reaction described previously. The solution was incubated in dark at room temperature for 1 h, quenched with 4× reducing Laemmli SDS sample loading buffer and boiled at 90° C. for 5 min. Samples were then separated by molecular weight on precast FuturePAGE 4-20% gels and imaged by ChemiDoc MP for measuring in-gel fluorescence. SimplyBlue™ SafeStain was applied to measure the protein loading.

### In-Gel Fluorescence Assay of Protein Labeling in Live HepG2 Cells by QMP-SO-C5-Alkyne

HepG2 cells were cultured on 90 mm dishes under a humidified atmosphere of 5% CO₂ at 37° C. in a complete DMEM medium. At 70% confluency, the cells were incubated in a complete DMEM medium containing QMP-SO-C5-alkyne (30 μM) and indicated concentrations of menadione in the presence or absence of NAC (5 mM) or MnTBAP (200 μM) for 4 h. For the MnTBAP-treated condition, cells were pre-incubated with MnTBAP for 12 h before the 4 h-treatment with QMP-SO-alkyne, menadione and MnTBAP. The cells were washed twice with PBS and lysed by sonication in PBS on ice. The protein concentrations were measured by BCA. 50 μL of the lysates were labeled with azide-fluor 545 by CuAAC reaction according to previously described procedures. The solution was incubated in dark at room temperature for 1 h, quenched with 4× reducing Laemmli SDS sample loading buffer and boiled at 90° C. for 5 min. Proteins were separated by molecular weight on precast FuturePAGE 4-20% gels and scanned by ChemiDoc MP for measuring in-gel fluorescence. SimplyBlue™ SafeStain was applied to measure the protein loading.

### MTT Cell Viability Assay of QMP-SO-C5-Alkyne

HepG2 cells were cultured on 96-well plates under a humidified atmosphere of 5% CO₂ at 37° C. in complete DMEM medium. At 70% confluency, the cells were then incubated with DMSO solvent control or QMP-SO-C5-alkyne at indicated concentrations for 4 h. After 4 h, 10 μL of MTT solution in PBS (5 mg/mL) was added to the cells for the final concentration of 0.25 mg/mL. The cells were incubated in dark at 37° C. with 5% CO₂ for 4 h, then lysed with 100 μL of SDS solution in PBS (0.5 g/mL with 0.01 M HCl). The plates were kept in dark overnight, and cell viability was assayed by measuring the absorption at 580 nm on Perkin Elmer Victor 3 (Molecular Devices).

Confocal Fluorescence Imaging of HepG2 Cells Labeled with QMP-SO-C5-Alkyne

HepG2 cells (3×10⁴ cells) were cultured on the 8-well Nunc Lab-Tek chambered slide system under a humidified atmosphere of 5% CO₂ in air at 37° C. 48 h in complete DMEM medium. At 70% confluency, the cells were incubated in a complete DMEM medium containing QMP-SO-C5-alkyne (30 μM) and indicated concentrations of menadione and in the presence or absence of MnTBAP (200 μM) or PEG-SOD (300 U/mL) for 4 h. For the experiments with MnTBAP and PEG-SOD, the cells were pre-incubated with MnTBAP or PEG-SOD for 12 h before the 4 h-treatment with QMP-SO-C5-alkyne, menadione and MnTBAP/PEG-SOD. After the treatment, cells were washed with PBS and fixed by pre-chilled methanol at −20° C. for 10 min. After fixation, the cells were washed with PBS and permeabilized with 0.3 vol % Triton X-100 at room temperature for 30 min. Next, cells were washed with PBS and incubated with CuAAC master-mix solution with CuSO₄, THPTA, azide-fluor 545 and sodium ascorbate at final concentrations of 100, 500, 20 and 5000 μM respectively. The cells were incubated in dark for 1 h, then washed with PBS and stained with Hoechst 33342 in PBS (8.2 μM) at room temperature for 15 min. The cells were washed thrice and then imaged in PBS by The Zeiss LSM880 with Airyscan 2 confocal microscope.

### Shotgun MS to Investigate Covalent Modification of BSA by QMP-SO-1

1 μL of QMP-SO-1 (50 mM) and 20 μL KO₂ (5 mM) were added to 80 μL of BSA (10 mg/mL) in PBS and incubated at 37° C. for 1 h. KO₂ was replaced with an equal volume of DMSO in the control sample. After incubation, proteins were precipitated with pre-chilled acetone (600 μL) at −20° C. overnight. The samples were centrifuged at maximum speed at 4° C. for 10 min, and the supernatant was discarded. The protein pellets were washed with pre-chilled 0.01M HCl/90% acetone, then resuspended in 100 μL of 8M urea in PBS. Protein concentration was measured with BCA and normalized to 1.67 mg/mL. 15 μL of the diluted samples was aliquoted out, allowing 25 μg of the proteins for further MS preparation. 20 μL of 1× ProteaseMax was added to the samples with vigorous vortex for 15 s. Then, 58.5 μL of ammonium bicarbonate (0.1 M) was added. The samples were then added with 10 μL of TCEP (110 mM) and incubated at 60° C. for 30 min. Next, 10 μL of iodoacetamide (IA, 150 mM) was added and the solution mixture was incubated at 37° C. for 30 min. After that, the samples were mixed with 1.2 μL of 5× ProteaseMax and vortexed. Sequencing-grade trypsin (20 μg; Promega) was reconstituted in 40 μL trypsin buffer and 1.5 μL of the trypsin solution was added to each sample. Samples were incubated at 37° C. overnight. Samples were then acidified with a final concentration of 5% formic acid and centrifuged at 13200 g for 30 min. The supernatant was collected, desalted using C18 StageTips, and sent for LC-MS/MS analysis on commercial C18 column (75 μm i.d.×50 cm length×2 μm particle size) coupled to a NanoTrap column (75 μm i.d.×2 cm length×3 μm particle size) with Orbitrap Fusion Tribid Lumos mass spectrometer (ThermoFisher).

Chromatographic separation was carried out using a linear gradient of increasing buffer B (80% MeCN and 0.1% formic acid) and declining buffer (0.1% formic acid) at 300 nL/min. Buffer B was increased to 27.5% B in 88 min and ramped to 44% B in the next 16 min, followed by a quick ramp to 95%. An isocratic gradient of 95% buffer B over 5 min, a decrease of buffer B to 3% and then the column was re-equilibrated. MS data was collected over a m/z range of 350-1500 m/z. A data-dependent top speed method with a time interval of 3s between every survey scan was operated during which higher-energy collisional dissociation (HCD) was used. Spectra were obtained at 30,000 MS2 resolution with a custom normalized AGC target of 200% and maximum ion injection time (IT) of 60 ms, 1.6 m/z isolation width, and normalized collisional energy of 30%. Preceding precursor ions targeted for HCD were dynamically excluded of 20 s.

The data was searched against the UniProt BSA database (UP00000913) using MaxQuant v2.0.3.0⁵³, specified with trypsin digestion (allowed up to 3 missed cleavages) and cysteine carbamidomethylation (+57.02146) as a static modification. The search also allowed up to 5 variable modifications for methionine oxidation (+15.99491), N-terminal acetylation (+42.01056), quinone methide modification (Cysteine, +49.072; Serine, threonine, aspartic acid, glutamic acid, histidine, tyrosine, arginine and lysine, +106.0419). The peptide false discovery rate (FDR) was set to 1%.

### QMP-SO-TMT Chemoproteomics Experiment

HepG2 cells were cultured on 150 mm dishes under a humidified atmosphere of 5% CO₂ at 37° C. for 48 h in complete DMEM medium. At cell confluency of 70%, the cells were incubated in complete DMEM medium containing QMP-SO-C5-alkyne (30 μM) and menadione (100 μM) for 4 h. Menadione was replaced with DMSO in the control sample. The cells were washed twice with PBS and lysed by sonication in PBS on ice. The protein concentrations were measured by BCA. A master-mix solution for the CuAAC reaction was prepared with CuSO₄, THPTA, DTB-PEG-azide and sodium ascorbate at the final concentrations of 1 mM, 3 mM, 100 μM and 5 mM, respectively. 360 μL of the CuAAC master mix solution was added to 2.5 mL of cell lysates (2 mg/mL) for DTB-PEG-azide conjugation through the CuAAC reaction. After 1 h incubation at room temperature, proteins were precipitated with pre-chilled acetone (18 mL) at −20° C. overnight. The samples were centrifuged at 5,000 g at 4° C. for 10 min, and the supernatant was discarded. The protein pellet was washed twice with methanol and resuspended in 1.2% SDS in PBS (w/v) with heating at 80° C. for 5 min. The samples were centrifuged, and the supernatant was transferred to a PBS solution containing Pierce™ Streptavidin Agarose beads (20349; Thermo Scientific) with a final concentration of SDS equal to 0.2% (w/v). The samples and beads were incubated at 4° C. with rotation overnight. The beads were then washed with PBS and water and re-dispersed in 6M urea in PBS. The samples were reduced by TCEP (1 mM) at 65° C. for 20 min, followed by alkylation with iodoacetamide (18 mM) at 37° C. for 30 min in dark. The beads were centrifuged at 1,400 g for 2 min, washed with PBS and re-suspended in 2M urea in PBS. The proteins on the beads were then digested by sequencing grade trypsin (Promega) at 37° C. overnight. After tryptic digestion, the beads were centrifuged at 1,400 g for 2 min, and the supernatant was collected. The beads were washed twice with 100 μL PBS, centrifuged, and supernatant collected. The combined fractions gave a total volume of 400 μL tryptic digested peptides. Samples were dried under a vacuum concentrator and redissolved in 70 μL TEAB (50 mM). TMT-6plex reagents were prepared according to the manufacturer's instructions. 30 μL acetonitrile and 1.5 μL of the TMT reagent in acetonitrile were added to each sample and the solution mixtures were incubated at room temperature for 1 h. Then, samples were added 0.6 μL of 5% hydroxylamine and incubated for 15 min. The resulting samples were mixed at equal volumes, desalted using C18 StageTips, and sent for LC-MS/MS analysis on commercial C18 column (75 μm i.d.×50 cm length×2 μm particle size) coupled to the Orbitrap Fusion Tribid Lumos mass spectrometer (Thermo Fisher).

Chromatographic separation was carried out using buffer A (0.1% formic acid) and buffer B (80% MeCN and 0.1% formic acid) with the flow rate of 300 nL/min. Buffer B was increase to 27.5% with a linear gradient in 164 min, followed by a further increase to 44% over 52 min. Then, there were a quick ramp of buffer B to 95% in 2 min, an isocratic gradient of 95% buffer B over 7 min and a quick decrease to 3%, where it was held and the column was re-equilibrated. The Orbitrap Fusion was operated in a data-dependent mode for both MS2 and MS3. MS1 scan was acquired in the Orbitrap mass analyzer with resolution 120,000 at m/z 400. Top speed instrument method was used for MS2 and MS3. For MS2, the isolation width was set at 0.5 Da and isolated precursors were fragmented by CID at a normalized collision energy (NCE) of 35% and analyzed in the ion trap using “turbo” scan. Following the acquisition of each MS2 spectrum, a synchronous precursor selection (SPS) MS3 scan was collected on the top 10 most intense ions in the MS2 spectrum. SPS-MS3 precursors were fragmented by higher energy collision-induced dissociation (HCD) at an NCE of 60% and analyzed using the Orbitrap at a resolution of 50,000.

The data was searched against the UniProt human database (UP000005640) using TMT10-MS3 workflow in MSFragger 3.8, specified with trypsin digestion (allowed up to 2 missed cleavages) and cysteine carbamidomethylation (+57.02146) as a static modification. The search also allowed up to 5 variable modifications for methionine oxidation (+15.9949) and N-terminal acetylation (+42.0106). TMT-6 reporter ions annotations were assigned with quantification at MS3. Proteins with zero reporter ion intensity for 2 out of the 3 replicate runs in either the control group or treated group were filtered out. The reporter ions ratios for other peptides were calculated, and peptides with invalid values were filtered out. Gene Ontology Cellular Compartment (GOCC) and PANTHER GO-Slim Biology Process of the identified proteins were determined using Perseus v2.0.9.0. and Panther 18.0.

### Data Availability

The mass spectrometry proteomics data have been deposited to the ProteomeXchange Consortium via the PRIDE partner repository with the dataset identifier PXD052283 and PXD052284.

### Example 2—In Vitro Reactivity and Selectivity of QMP-SOs Toward Superoxide

This example aims to evaluate the in vitro reactivity, kinetic profile, and selectivity of various QMP-SO probes toward superoxide, in comparison with other reactive oxygen and nitrogen species (ROS/RNS). The objective is to validate the capability of QMP-SO probes to be selectively activated by superoxide under biologically relevant conditions, supporting their application in proximity labeling and redox proteomics.

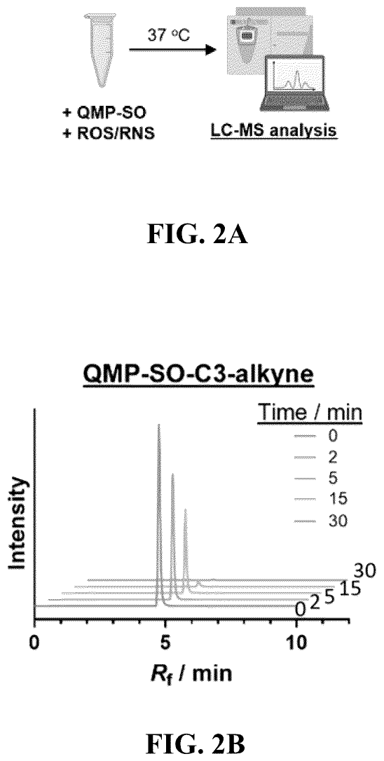

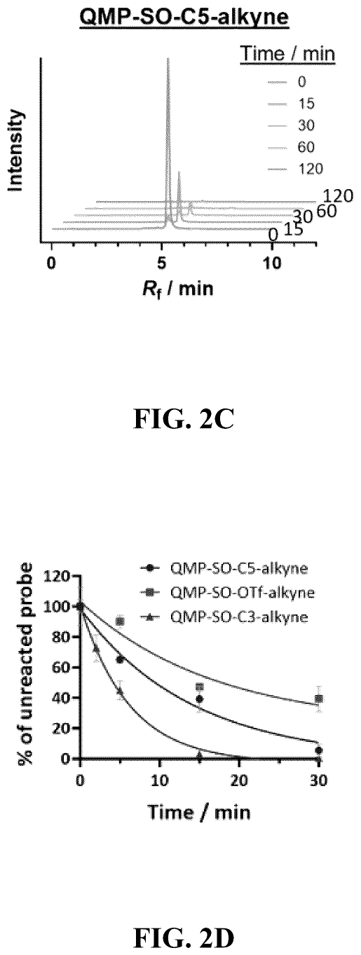

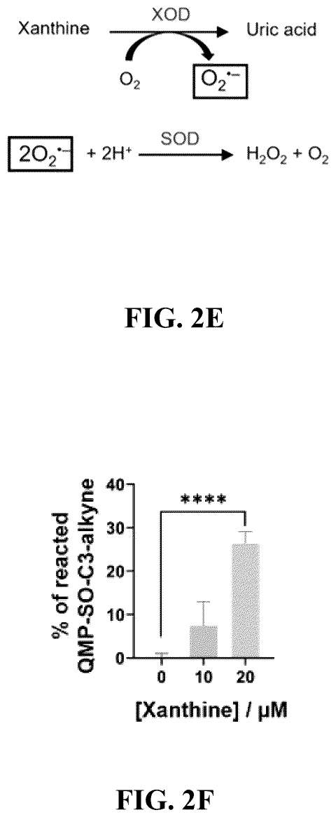

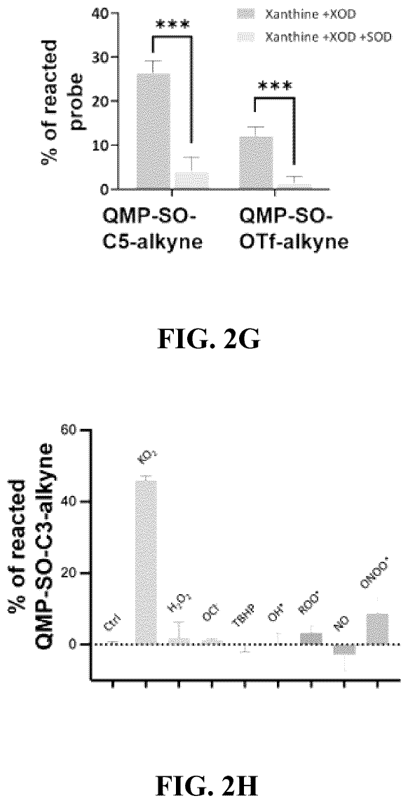

LC-MS was employed to monitor the consumption of QMP-SO probes in aqueous buffer systems upon exposure to superoxide. As shown in FIG. 2A, QMP-SO-C3-alkyne and QMP-SO-C5-alkyne exhibited rapid consumption in the presence of potassium superoxide (KO₂), with complete depletion observed within 15 and 60 minutes, respectively (FIGS. 2B-2D). In contrast, QMP-SO-OTf-alkyne, which contains a triflate trigger instead of diphenylphosphonate, reacted more slowly under identical conditions. Furthermore, enzymatically generated superoxide (via the xanthine/xanthine oxidase system) also induced pronounced depletion of QMP-SO-C3-alkyne and QMP-SO-C5-alkyne (FIGS. 2E-2G), again exceeding the response observed with QMP-SO-OTf-alkyne (FIGS. 2F-2G). These results suggest that QMP-SOs with a diphenylphosphonate trigger should be more sensitive than those with a triflate group for superoxide detection.

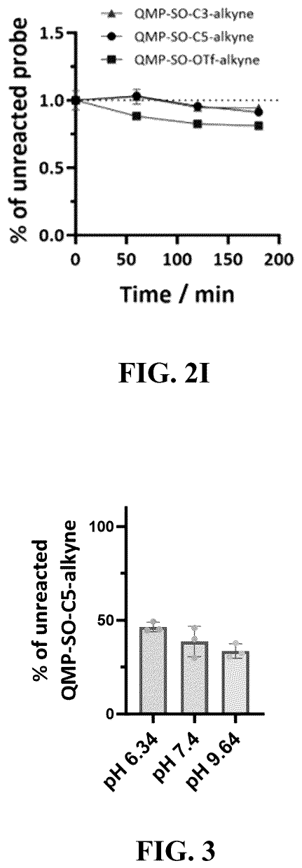

The superoxide-specificity of the probes was further confirmed by co-incubation with SOD, a known enzymatic scavenger of superoxide. The presence of SOD significantly reduced the consumption of QMP-SOs (FIGS. 2E-2G), demonstrating that the observed probe activation was indeed attributable to superoxide. Additionally, pH variation in the reaction buffer did not markedly affect the reactivity of QMP-SO-C5-alkyne with superoxide (FIG. 3), supporting the robustness of the probes under physiological conditions.

To assess selectivity, QMP-SO-C3-alkyne was exposed to other ROS/RNS including hydroxyl radicals (·OH), hypochlorite (OCl⁻), tert-butoxyl radicals ((CH₃)₃COO·), nitric oxide (NO·), and peroxynitrite (ONOO⁻). As shown in FIG. 2H, negligible probe consumption was observed for most species, except for a mild reactivity with peroxynitrite. Nevertheless, the extent of peroxynitrite-mediated probe depletion was significantly lower—less than 20% of that observed with superoxide—despite being tested at supraphysiological concentrations (100 μM). This suggests minimal interference from peroxynitrite in biological applications. In control experiments, the probes remained chemically stable in the absence of superoxide (FIG. 2I), indicating favorable shelf-life and handling characteristics.

These results collectively demonstrate that QMP-SO probes, particularly those incorporating a diphenylphosphonate trigger, react rapidly and selectively with superoxide under physiological conditions, with minimal off-target activation by other ROS/RNS. This validates their use as reliable tools for superoxide-responsive covalent protein labeling in chemoproteomic and imaging applications.

### Example 3—Superoxide-Dependent Protein Labeling by QMP-SO Probes In Vitro

This example aims to evaluate the ability of QMP-SO probes to covalently label proteins in a superoxide-dependent manner under in vitro conditions. BSA was selected as a model protein to assess both the chemical reactivity and the labeling specificity of the probes toward nucleophilic amino acid residues.

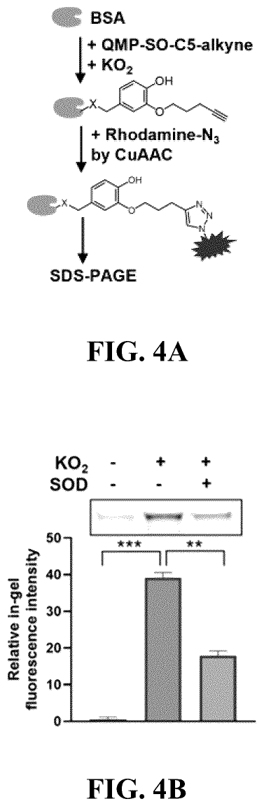

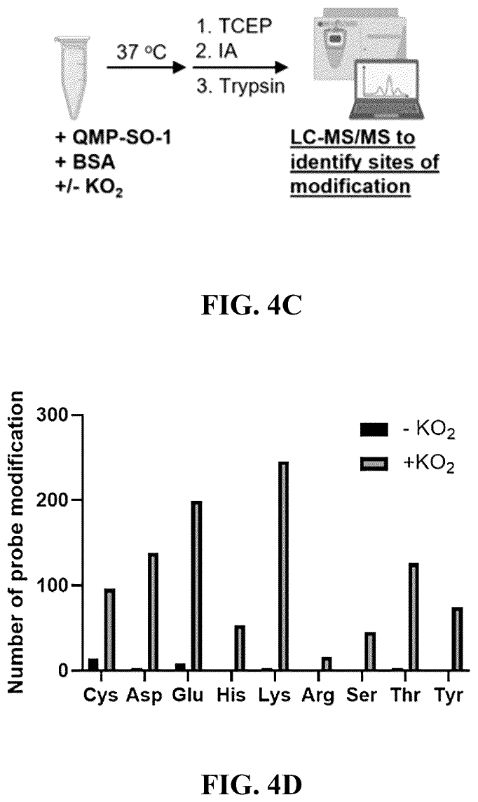

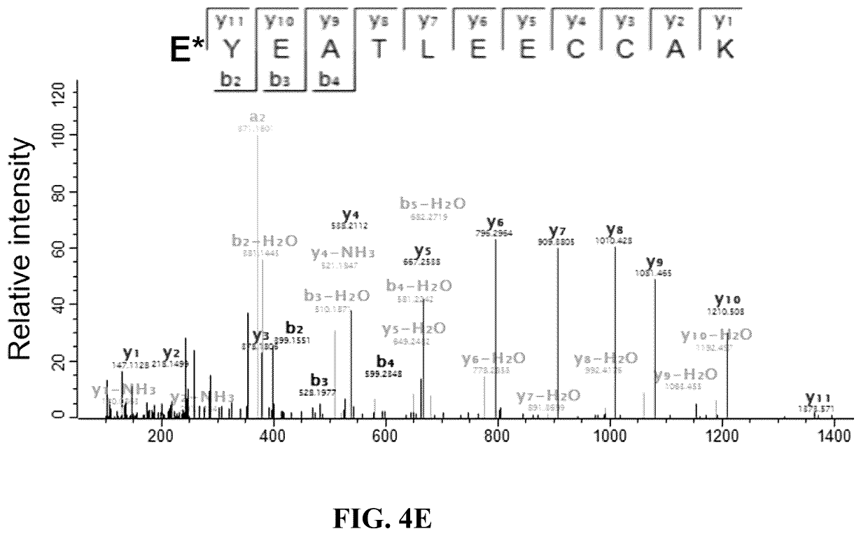

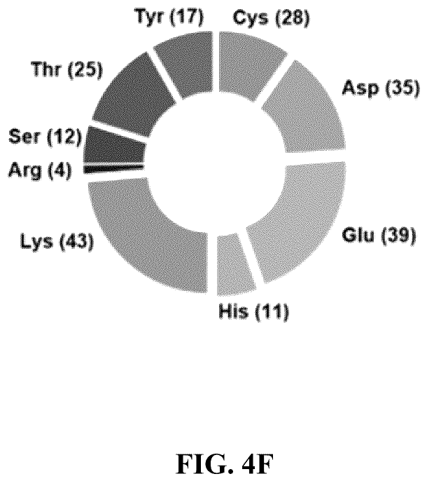

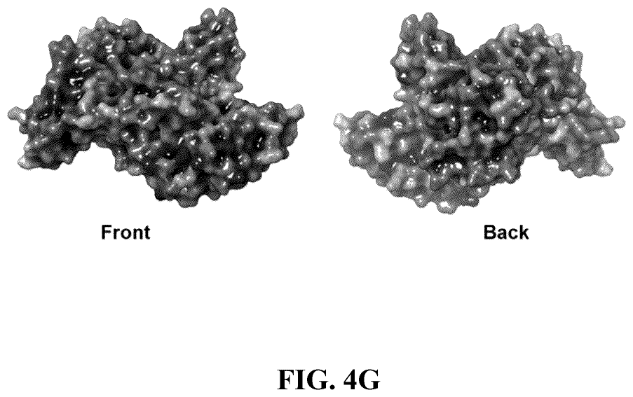

Following the confirmation of rapid reactivity between QMP-SOs and superoxide (see Example 2), the covalent tagging capability of QMP-SOs toward proteins was examined. An aqueous solution of BSA was incubated with QMP-SO-C5-alkyne in the presence or absence of KO₂ and/or SOD. The reaction mixtures were then subjected to CuAAC reaction using a fluorophore-azide, enabling fluorescent labeling of probe-modified proteins. As shown in FIG. 4A, strong in-gel fluorescence was observed when BSA was treated with both QMP-SO-C5-alkyne and KO₂, indicating effective protein tagging. In contrast, co-incubation with SOD significantly reduced the fluorescence signal (FIG. 4B), confirming that the labeling was dependent on superoxide.

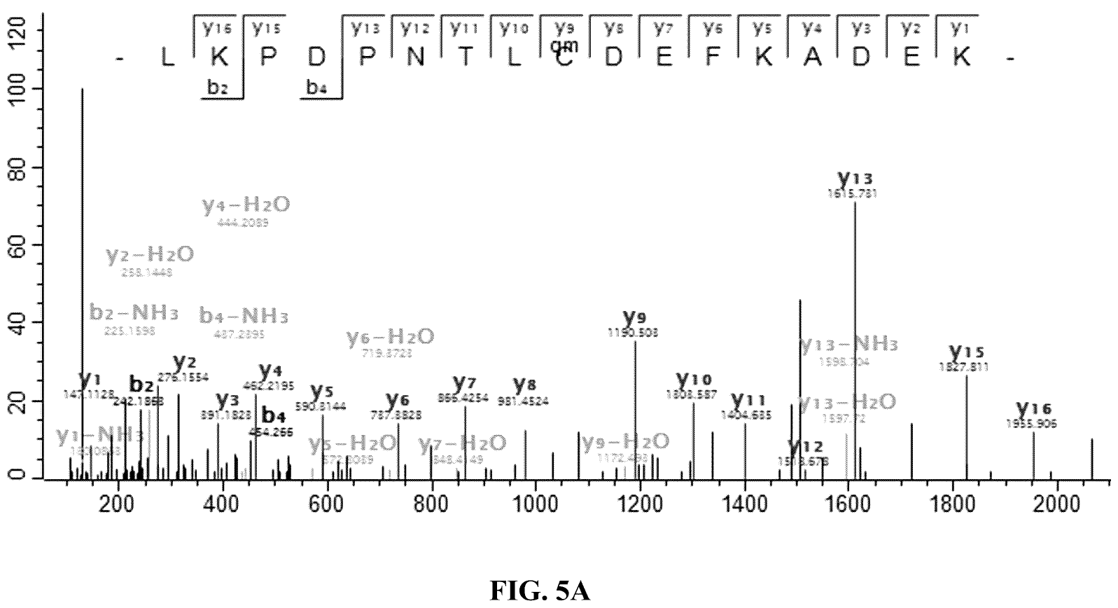

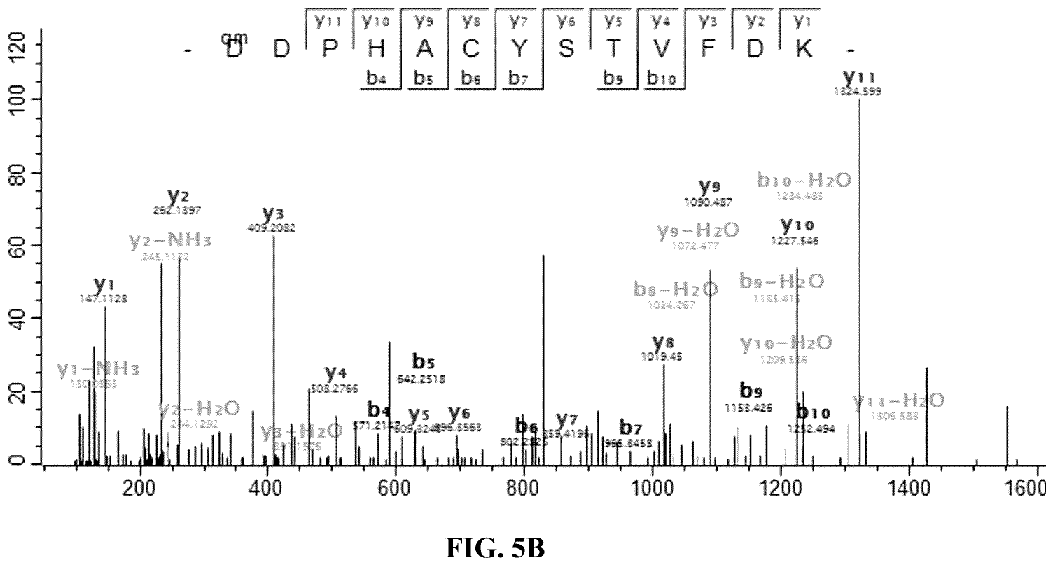

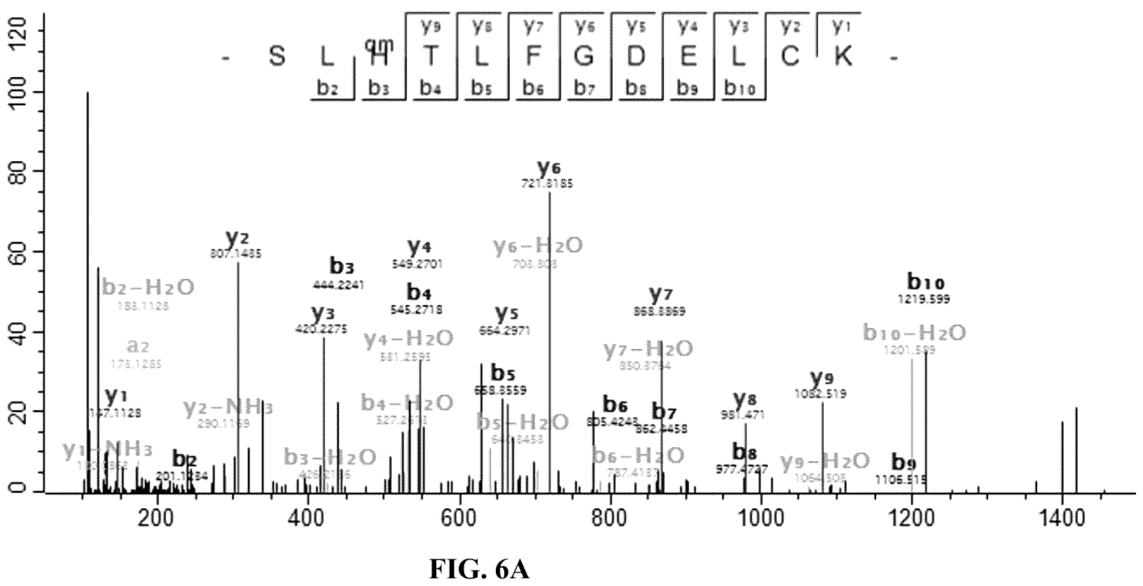

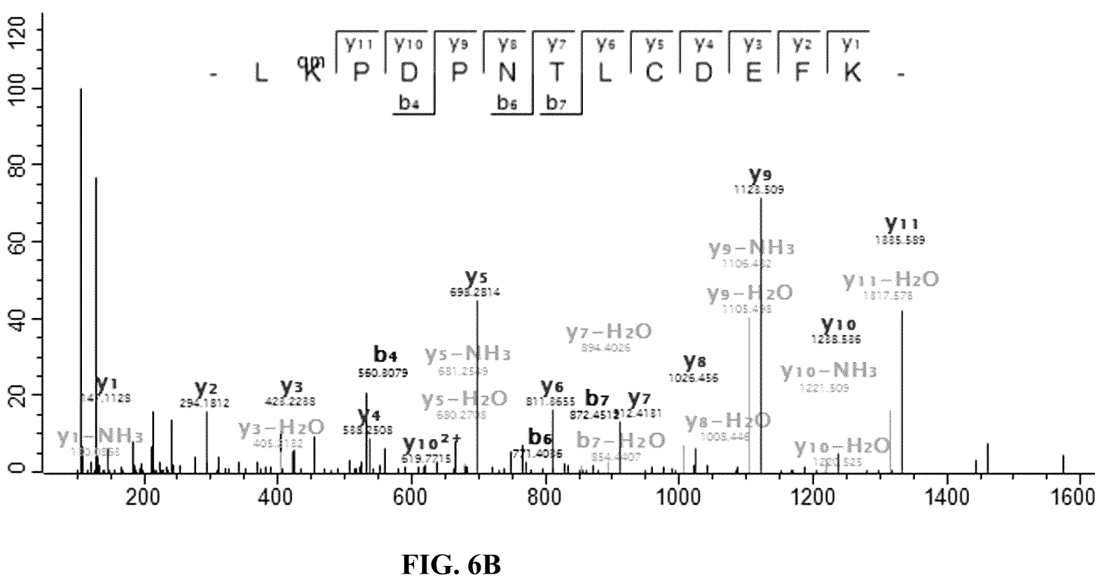

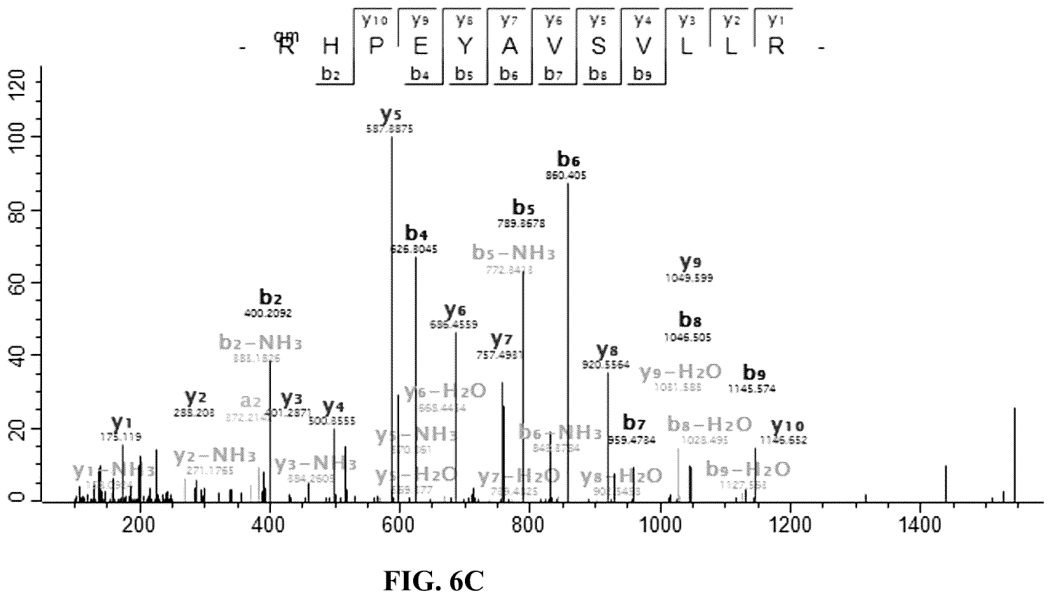

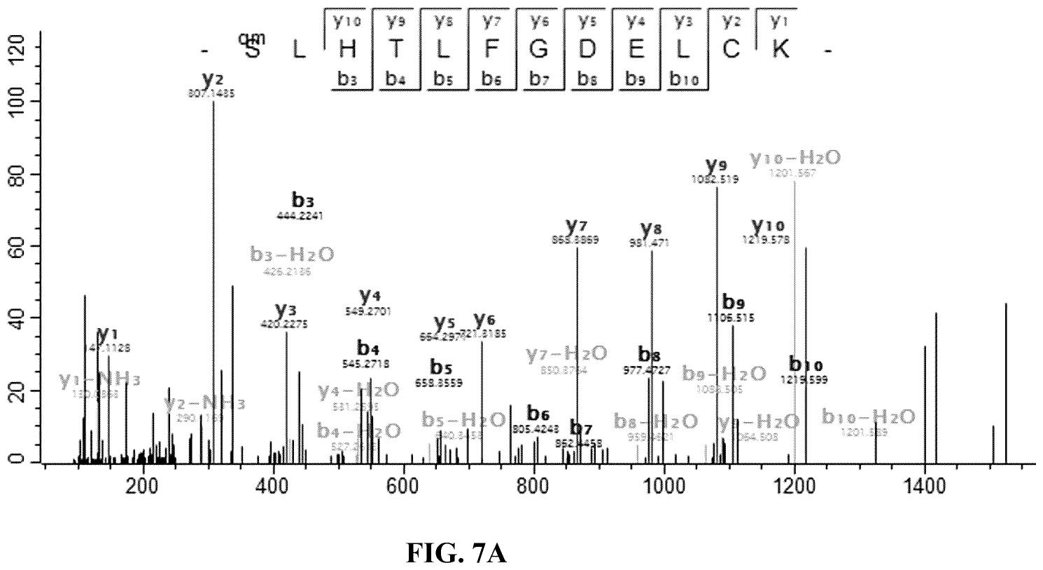

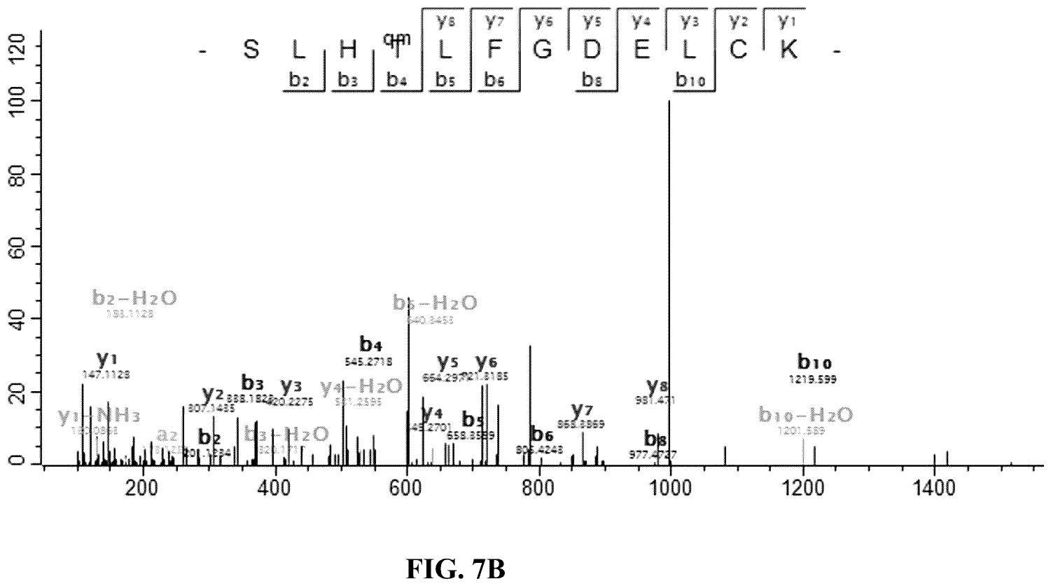

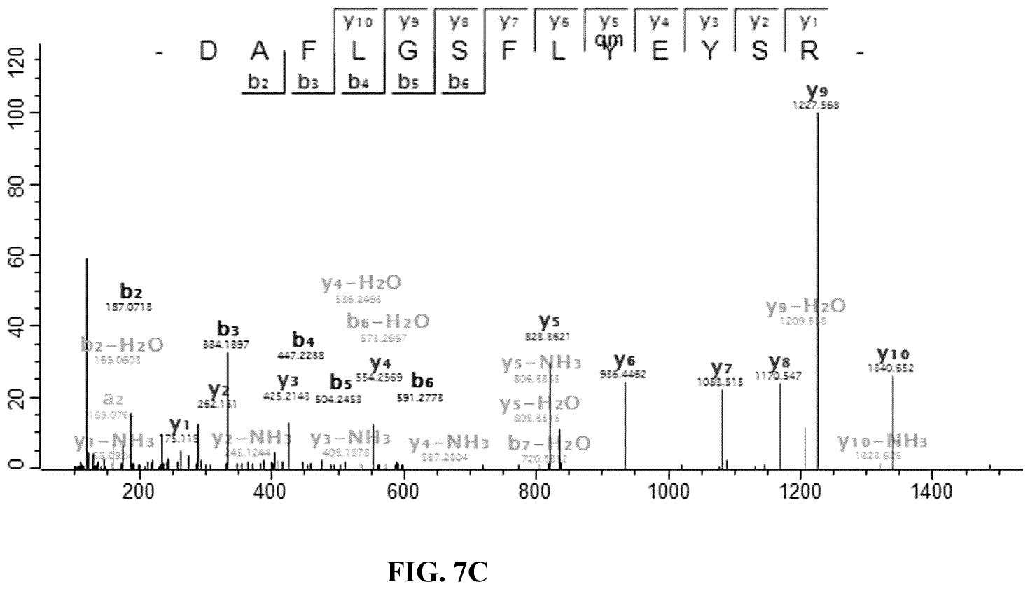

To identify the covalent modification on BSA by QMP-SOs, LC-MS/MS analysis was performed on tryptic digests of BSA incubated with QMP-SO-1 and KO₂. As shown in FIG. 4C, multiple covalent modifications bearing a 4-(hydroxyphenyl)methylene adduct mass were detected on various nucleophilic amino acid residues, including Cys, Asp, Glu, His, Lys, Arg, Ser, Thr, and Tyr (FIGS. 4D-4E and FIGS. 5-7). These findings support a reaction mechanism wherein QMP-SOs undergo superoxide-triggered elimination to generate quinone methide intermediates, which then undergo electrophilic addition to protein side chains (FIG. 1C). Notably, the presence of KO₂ led to a >27-fold increase in the number of detected modification sites (FIG. 4D), highlighting the superoxide-dependence of the covalent labeling process.

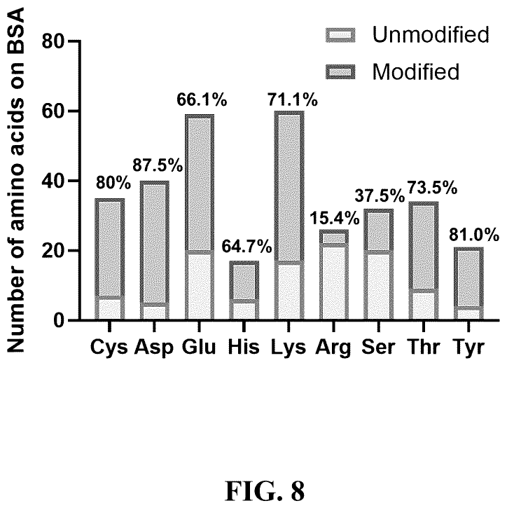

The identified labeling sites were broadly distributed across nucleophilic residues (FIGS. 4F and 8), with a predominant localization on surface-exposed regions of the protein (FIG. 4G). This broad reactivity with amino acids illustrates that QMP-SO should allow good tagging on almost all possible proteins of interest in a superoxide-dependent manner, regardless of the protein primary sequence. This is an important feature for profiling proteins associated with superoxide biology.

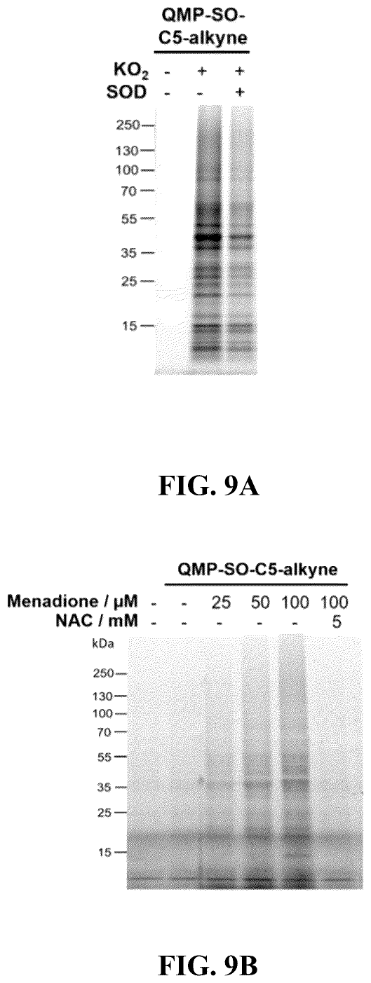

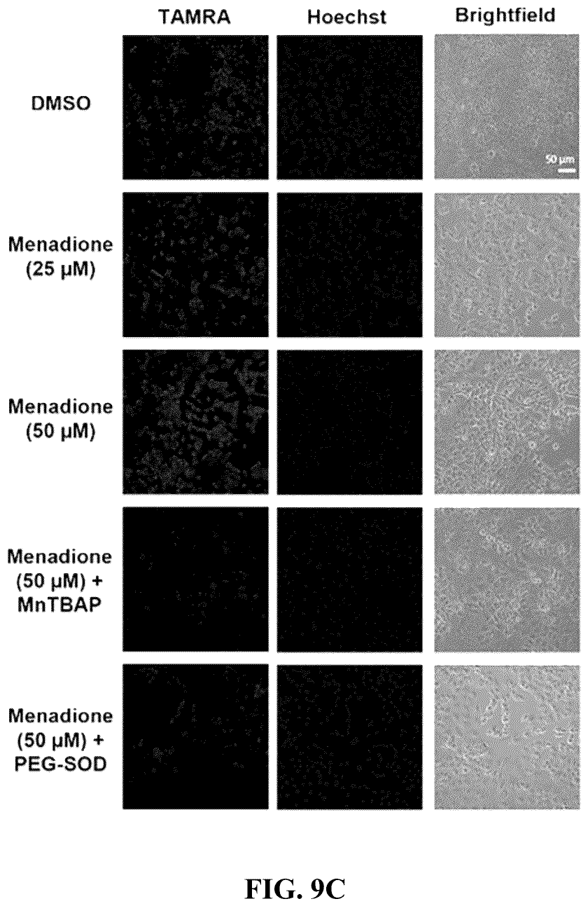

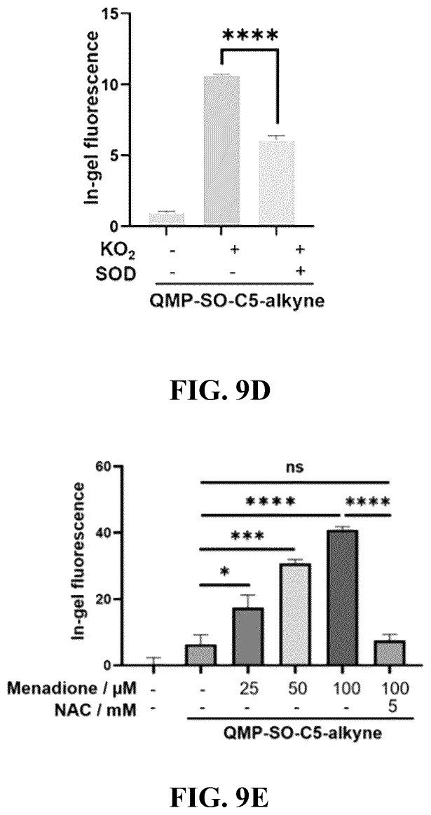

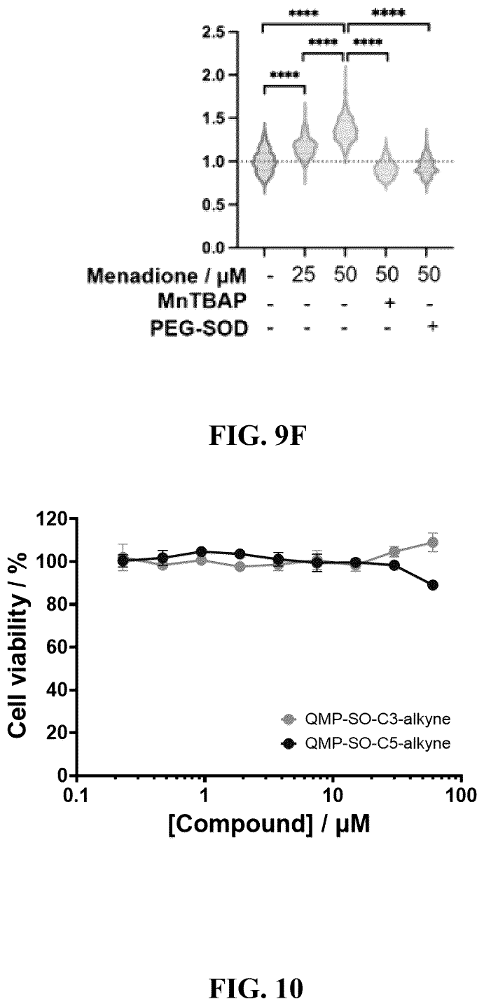

To further validate the utility of QMP-SO probes in complex biological samples, the labeling of proteins in HepG2 cell lysates was investigated. As shown in FIGS. 9A-9D, incubation of the lysates with QMP-SO-C5-alkyne and KO₂ induced a marked increase in in-gel fluorescence, indicative of successful protein tagging. Addition of SOD substantially diminished the signal, reaffirming the superoxide-specific nature of the probe reactivity. This indicates successful superoxide-induced tagging of proteins by QMP-SO-C5-alkyne, as well as the high specificity of QMP-SO-C5-alkyne toward superoxide.

### Example 4—Fluorescence Imaging-Based Detection of Superoxide Detection in Cancer Cells by QMP-SOs

This example aims to evaluate the application of QMP-SO probes in live-cell imaging for detecting intracellular superoxide production. The objective is to determine whether QMP-SO-C5-alkyne can selectively respond to endogenous superoxide in cancer cells and enable its visualization by in-gel and confocal fluorescence imaging.

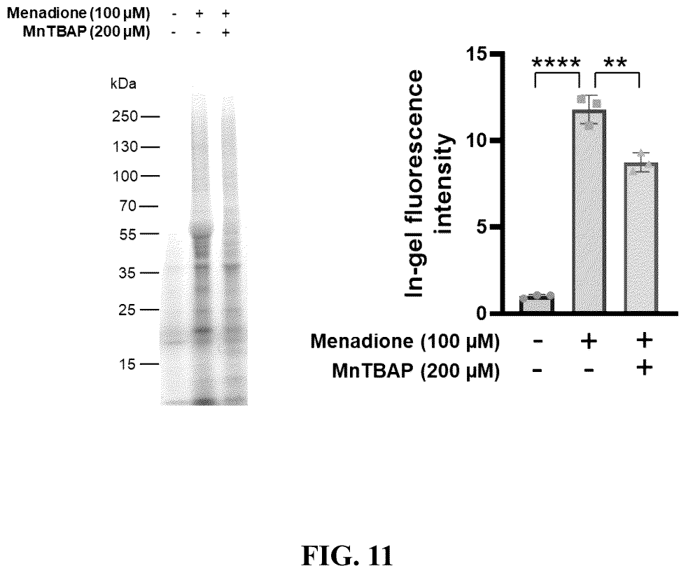

Live HepG2 hepatocellular carcinoma cells were treated with QMP-SO-C5-alkyne in the presence or absence of menadione and/or NAC. Menadione is known to induce endogenous production of superoxide, particularly in mitochondria. Following incubation, cells were lysed by probe sonication, and the resulting protein lysates were subjected to CuAAC labeling with azide-fluor 545, followed by SDS-PAGE and in-gel fluorescence analysis. As shown in FIGS. 9B and 9E, a dose-dependent increase in in-gel fluorescence intensity was found in HepG2 cells co-treated with menadione and QMP-SO-C5-alkyne, indicating enhanced protein labeling under elevated superoxide conditions. Co-incubation with the antioxidant NAC abolished the fluorescence enhancement. This can be explained by the high local concentrations of superoxide generated in HepG2 cells upon menadione treatment, resulting in superoxide-mediated covalent tagging of proximal proteins by QMP-SO-C5-alkyne. It was confirmed that QMP-SO-C5-alkyne was not toxic to HepG2 cells during the 4 h incubation, with >90% viable cells at the 40 μM treatment as revealed by the MTT assay (FIG. 10). Also, a diminished in-gel fluorescence intensity was found in cells treated with the SOD mimetic, MnTBAP⁴⁴ (FIG. 11), further validating the probe's specificity for superoxide.

To further investigate the spatial distribution of superoxide-related protein labeling, confocal fluorescence microscopy was performed. Live HepG2 cells were incubated with QMP-SO-C5-alkyne and treated with menadione, MnTBAP (SOD mimetic) and/or cell-permeable PEG-SOD. The treated cells were fixed by pre-chilled methanol and permeabilized by PBS with 0.3 vol % Triton-X100. The probe-labeled proteins were then reacted with azide-fluor 545 through CuAAC reaction, and the fixed cells were stained with Hoechst and imaged by confocal microscopy. As illustrated in FIGS. 9C and 9F, a concentration-dependent increase in intracellular fluorescence was observed upon menadione treatment, whereas both MnTBAP and PEG-SOD treatments significantly suppressed the fluorescence intensity. This illustrates that the QMP-SO-induced protein labeling is superoxide-dependent and QMP-SO is highly sensitive to dynamic changes in cellular superoxide levels, enabling superoxide monitoring by confocal fluorescence imaging.

### Example 5—QMP-SO-Enabled Chemoproteomics Profiling of Proteins Associated with Superoxide Redox Biology

This example aims to demonstrate the application of QMP-SO-based chemoproteomics to identify and profile endogenous proteins that are selectively modified under superoxide-rich conditions in live cells. Given that excessive superoxide production can modulate cell survival, stress response, and metabolism, this study seeks to uncover the protein targets and signaling pathways regulated by superoxide through covalent labeling with QMP-SO probes and tandem mass tag (TMT)-based mass spectrometry.

HepG2 cells can withstand oxidative stress induced by menadione treatment at a low dosage/for a short period of time, while higher doses/prolonged incubation results in substantial cell death. However, the proteins that govern the cellular signals for survival, metabolism and cell death under menadione treatment remain underexplored.

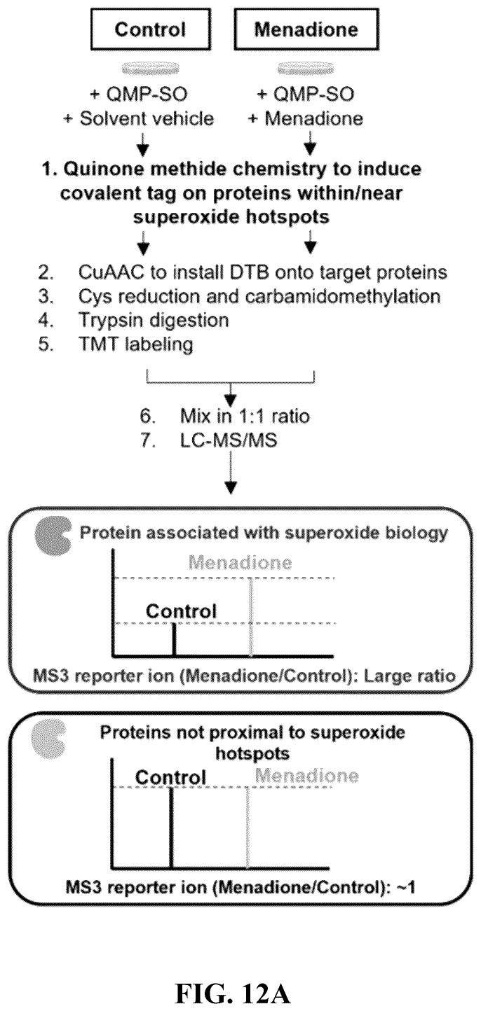

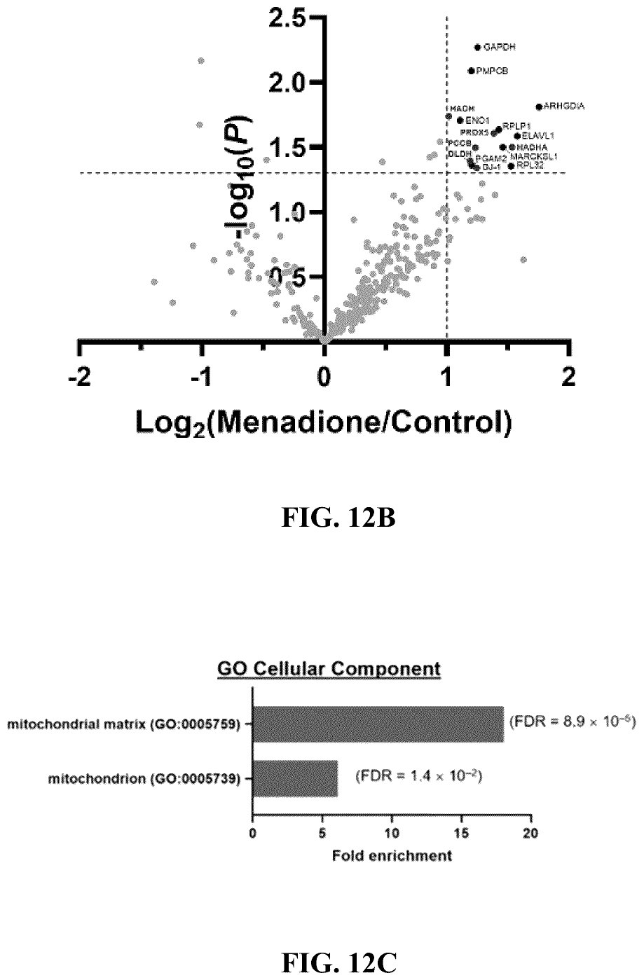

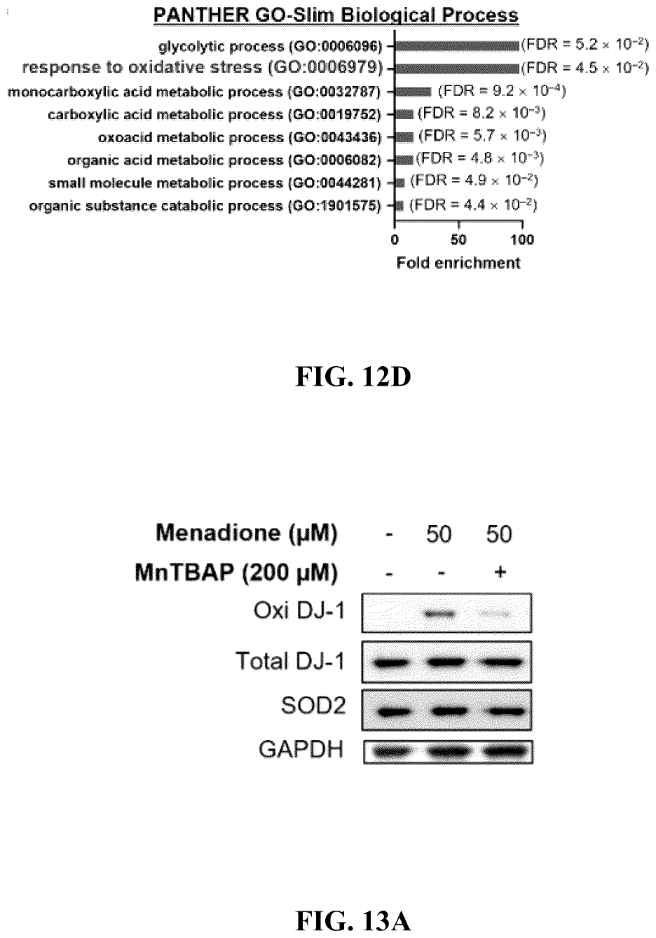

HepG2 cells were treated with menadione to induce intracellular superoxide production. To identify superoxide-responsive proteins, a-based chemoproteomics workflow was developed using QMP-SO-C5-alkyne as the covalent labeling probe (FIG. 12A). The workflow involves probe treatment, CuAAC-based biotin conjugation, streptavidin enrichment, trypsin digestion, and TMT labeling followed by LC-MS/MS analysis.

Based on the high reactivity and small diffusion radius of superoxide, this platform enables selective enrichment of proteins localized in superoxide-abundant subcellular regions (“superoxide hotspots”). By using QMP-SO-C5-alkyne to induce a covalent tag onto proximal proteins in superoxide hotspots, we successfully enriched and profiled 15 proteins with a 2-fold enrichment and statistical significance in the menadione-treated samples (FIG. 12B). GO cellular component analysis revealed predominant localization of these proteins in mitochondria and the mitochondrial matrix (FIG. 12C), aligning with known sites of superoxide generation under redox stress.

More importantly, these proteins were associated with the response to oxidative stress (FIG. 12D), and 6 of them have been annotated to the oxidation-reduction process (GOBP: 0055114; FIG. 12B). All these results highlight the success of QMP-SO-C5-alkyne in tagging proteins within or near superoxide hotspots to profile proteins associated with redox biology by the TMT-based MS experiments.

### Example 6—Functional Analysis of Superoxide-Responsive Proteins DJ-1 and DLDH Identified by QMP-SO Chemoproteomics

This example aims to investigate the functional consequences of superoxide-mediated redox modifications on two key protein targets, DJ-1 and DLDH, identified through QMP-SO-C5-alkyne-enabled TMT chemoproteomic profiling.

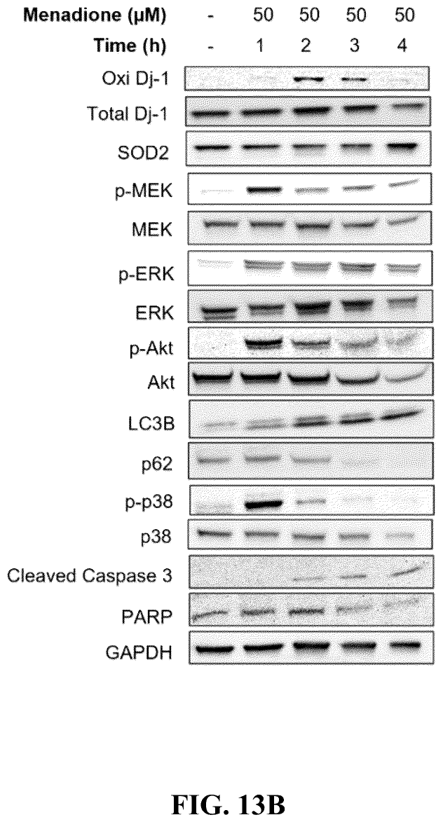

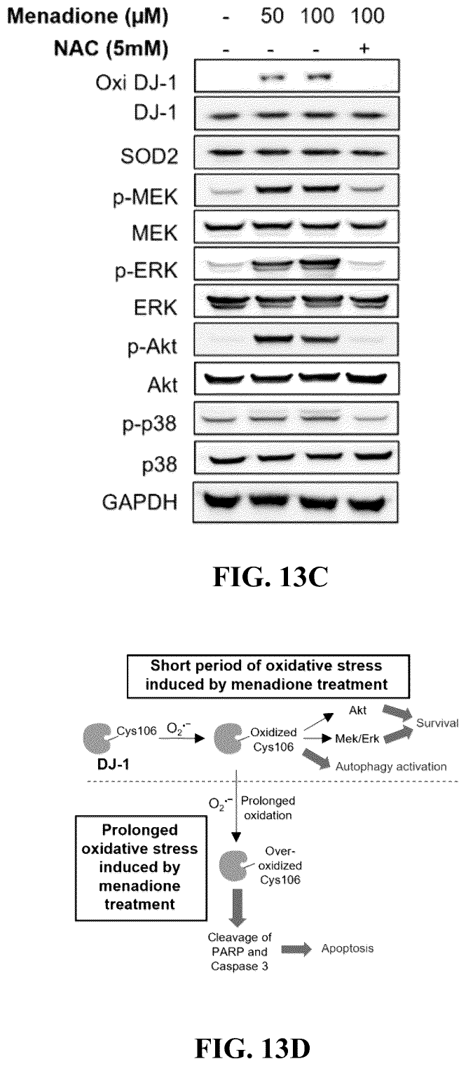

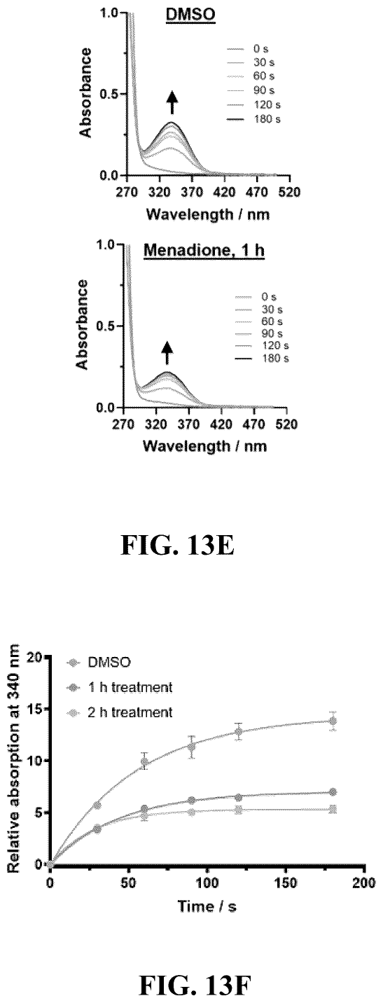

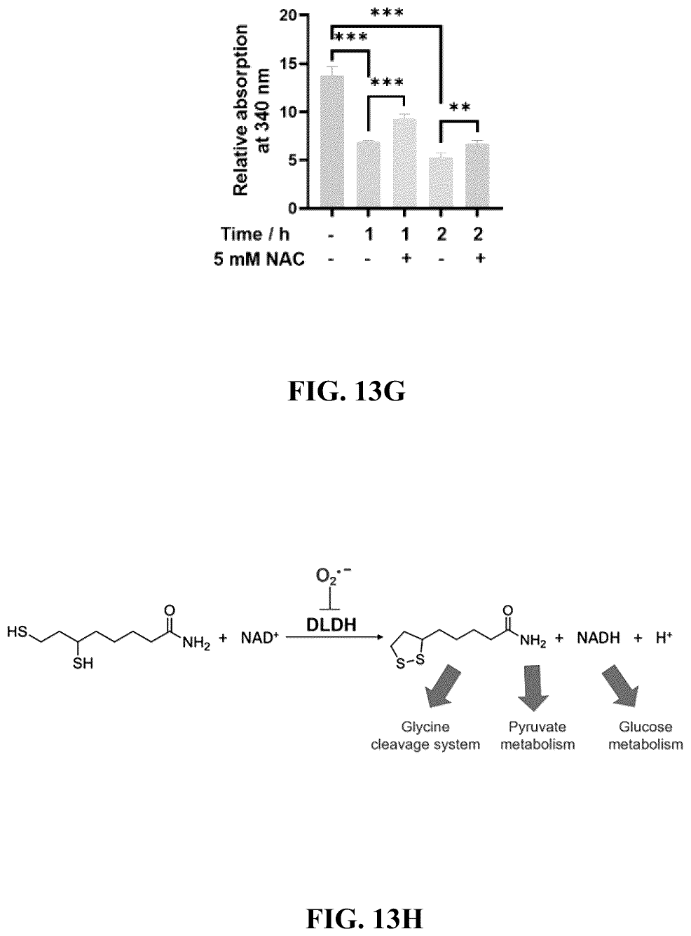

DJ-1 (PARK7) is one of the enriched proteins in the QMP-TMT experiment. It is known to activate proliferative signals through Erk1/2 and PI3K/Akt pathways and modulate the autophagy process. In HepG2 cells treated with 50 μM menadione for 2 hours, immunoblotting with an oxidation-specific antibody revealed increased sulfenylation/sulfonylation at Cys106 of DJ-1 (FIG. 13A). Co-treatment with the SOD mimetic, MnTBAP, resulted in a significant decrease in oxidized DJ-1 signal (FIG. 13A), suggesting that the oxidation of DJ-1 should be primarily mediated by superoxide. Menadione treatment also led to elevated phosphorylation of MEK, Erk1/2, and Akt (FIG. 13B), indicating activation of prosurvival signaling cascades.

Simultaneously, autophagy activation was observed, as evidenced by a decrease in p62 protein level and an increase in LC3B-II/LC3B-I ratio (FIG. 13B). It is noteworthy that co-incubation of cells with antioxidant NAC could recuse cells from all these changes induced by menadione (FIG. 13B). This illustrates the importance of oxidative modifications on DJ-1 in activating the pro-survival and autophagy signals. On the other hand, a longer treatment with menadione resulted in a decrease in DJ-1 Cys106 sulfenylation/sulfonylation (FIG. 13C), suggesting overoxidation and hence inactivation of DJ-1. This led to a decrease in pro-survival signal, as evidenced by the decrease in phosphorylation levels of MEK, Erk1/2 and Akt (FIG. 13C). A decrease in PARP level was also found in cells treated with menadione for 4 h, pointing to its cleavage and an induction of apoptosis. These results demonstrate the critical role of oxidative modifications and DJ-1 activity in governing pro-survival and pro-apoptotic events in cells treated with menadione, with superoxide being one of the primary ROS mediating these oxidative modifications (FIG. 13D).

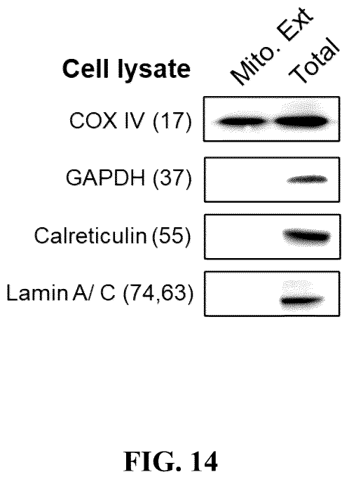

Dihydrolipoamide dehydrogenase (DLDH) is another protein target identified in the MS-based chemoproteomics experiment (FIG. 12B). It is a redox-active enzyme involved in energy metabolism as a component of the glycine cleavage system and as an E3 subunit in both the mitochondrial pyruvate dehydrogenase (PDH) and α-ketoglutarate dehydrogenase (α-KGDH) complexes. To evaluate the functional impact of superoxide on DLDH, mitochondrial extracts were prepared from HepG2 cells treated with either solvent vehicle or menadione (FIG. 14). Enzymatic activity was assessed by measuring the production of NADH from the oxidation of dihydrolipoamide in the presence of NAD⁺. A significant increase in UV-vis absorption at 340 nm over time was observed, owing to the production of NADH from the enzymatic reaction of DLDH (FIG. 13E). Notably, mitochondrial extracts from cells treated with menadione showed a slower increase in UV-vis absorption at 340 nm compared to the control sample (FIGS. 13F-13G), indicating the lower DLDH activity in extracts from menadione-treated cells. Notably, co-treatment with NAC partially restored DLDH activity (FIG. 13G), suggesting that the loss of function was due to reversible oxidative modification. Since DLDH is a confirmed target of QMP-SO-C5-alkyne and contains known redox-sensitive cysteines (Cys45 and Cys50) within its active site, the observed inhibition is likely attributable to superoxide-mediated oxidation (FIG. 13H).

Altogether, the QMP-SO probes offer a chemically precise and biologically informative platform for superoxide detection, imaging, and proteome-wide target profiling. Distinct from previously reported H₂O₂-responsive probes that undergo 1,4-elimination to form ortho-quinone methides, the present invention utilizes 1,6-elimination to generate para-quinone methides selectively upon superoxide reaction. This modular design supports customization with targeting groups for subcellular resolution. The invention opens new avenues for mechanistic investigation of redox biology and reveals the relevance of superoxide signaling in cellular processes such as proliferation, autophagy, and metabolism.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### INDUSTRIAL APPLICATION

By integrating mass spectrometry and chemoproteomic approaches, the QMP-SO probe enables the identification of proteins regulated by superoxide-mediated redox biology, revealing their involvement in critical cellular processes such as proliferation, apoptosis, metabolism, and autophagy. Given the central role of oxidative stress in various pathologies including cancer, aging, and neurodegenerative diseases, these superoxide-responsive proteins represent promising targets for future therapeutic development. The QMP-SO platform is therefore anticipated to be broadly applicable across diverse biological models to advance the discovery of redox signaling mechanisms and disease-relevant biomarkers.

1. A composition comprising a superoxide-reactive probe of formula (I):
a superoxide-specific quinone methide precursor (QMP-SO)-R¹, or a structural analog, derivative, or pharmaceutically acceptable salt thereof;
wherein QMP-SO comprises a quinone methide precursor moiety having a phenol ring substituted at a para-position with a superoxide-reactive trigger group, and a hydroxyl group of the phenol ring is covalently bonded to a linker;
wherein the linker comprises the R¹ selected from an alkyne group, an azide group, a biotin, or a fluorophore; and
wherein the composition is configured to generate a para-quinone methide intermediate upon reaction with superoxide under aqueous physiological conditions, and the para-quinone methide intermediate is capable of covalently labeling nucleophilic residues on nearby proteins.

2. The composition of claim 1, wherein the R¹ is an alkyne group suitable for Cu(I)-catalyzed azide-alkyne cycloaddition (CuAAC) reaction.

3. The composition of claim 1, wherein the superoxide-reactive trigger group comprises diphenylphosphonate or triflate.

4. The composition of claim 1, wherein the quinone methide intermediate selectively reacts with nucleophilic amino acid residues selected from the group consisting of cysteine, lysine, histidine, tyrosine, serine, threonine, glutamic acid, and aspartic acid.

5. The composition of claim 1, wherein the superoxide-reactive probe exhibits minimal reactivity toward other reactive oxygen or nitrogen species, including hydrogen peroxide, hypochlorous acid, nitric oxide, and peroxynitrite.

6. The composition of claim 1, further comprising a pharmaceutically acceptable carrier suitable for delivery to cells or tissues.

7. The composition of claim 1, wherein the R¹ is conjugated to a reporter moiety selected from a fluorophore or desthiobiotin via CuAAC.

8. A method for labeling superoxide-associated proteins in a biological sample, comprising:
contacting the biological sample with a composition of claim 1, wherein a superoxide-reactive probe in the composition is present in a concentration in a range of about 0.1 μM to 100 μM;
reacting the superoxide-reactive probe with endogenously or exogenously generated superoxide to form a quinone methide intermediate; and
covalently labeling proteins proximal to superoxide hotspots via electrophilic addition of the quinone methide intermediate to nucleophilic amino acid residues.

9. The method of claim 8, further comprising subjecting the labeled proteins to Cu(I)-catalyzed azide-alkyne cycloaddition (CuAAC) reaction with a detection reagent.

10. The method of claim 9, wherein the detection reagent comprises a fluorophore or a biotin moiety.

11. The method of claim 8, wherein the biological sample comprises a population of cells, tissue, an organoid, or a cell lysate derived from a mammal.

12. The method of claim 8, further comprising treating the biological sample with a superoxide-inducing agent selected from menadione, antimycin A, or xanthine/xanthine oxidase.

13. The method of claim 8, wherein the labeled proteins include DJ-1, and a residue of Cys106 of the DJ-1 is oxidatively modified in a superoxide-dependent manner.

14. The method of claim 8, wherein the labeled proteins include dihydrolipoamide dehydrogenase (DLDH), and the labeling occurs at one or more nucleophilic residues within DLDH, resulting from a superoxide-triggered para-quinone methide reaction.

15. The method of claim 8, wherein the biological sample is further treated with an antioxidant selected from N-acetylcysteine (NAC) or MnTBAP.

16. A kit for detecting superoxide-associated protein labeling, comprising:
the composition of claim 1;
a detection reagent comprising a fluorophore or biotin moiety; and
instructions for performing superoxide-dependent protein labeling.

17. The kit of claim 16, further comprising:
a superoxide-inducing agent selected from menadione or antimycin A; and
a superoxide scavenger selected from MnTBAP or N-acetylcysteine.

18. The kit of claim 16, wherein the detection reagent comprises a fluorophore or desthiobiotin moiety conjugated to an azide-functionalized polyethylene glycol (PEG) linker, formulated in an aqueous buffer comprising a copper(I)-stabilizing ligand selected from tris(benzyltriazolylmethyl)amine (TBTA) or bathophenanthroline disulfonic acid (BPDS), thereby enabling efficient Cu(I)-catalyzed azide-alkyne cycloaddition (CuAAC) under physiological pH and temperature conditions.

19. The kit of claim 16, further comprising a lyophilized control reagent comprising a purified protein covalently labeled at a defined nucleophilic residue by the superoxide-reactive probe, and packaged with reference SDS-PAGE and/or mass spectrometry data.

20. The kit of claim 16, wherein the superoxide-reactive probe is provided at a concentration of 0.1 μM to 100 μM; the detection reagent is provided at a concentration of at least 1 μM, and the kit components are arranged in a multi-well plate or vial-based format.
